(19) United States
(12) Patent Application Publication
(10) Pub. No.: US 2008/0293749 A1
(43) Pub. Date: Nov. 27, 2008

(54) Pyrrolidine(thi)ones Substituted by Heterocyclic Substituents in The 3-Position

(75) Inventors: Sven FRORMANN, Aachen (DE); Stefanie FROSCH, Aachen (DE); Carsten GRIEBEL, Aachen (DE); Derek SAUNDERS, Aachen (DE); Fritz THEIL, Berlin (DE); Heinz GRAUBAUM, Erkner (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 12/131,574

(22) Filed: Jun. 2, 2008

Related U.S. Application Data
(63) Continuation of application No. PCT/EP2006/011440, filed on Nov. 29, 2006.

(30) Foreign Application Priority Data
Dec. 2, 2005 (DE) 10 2005 057 912.4

(51) Int. Cl. A61K 31/517; A61P 29/00; A61P 37/00; C07D 403/04

(52) U.S. Cl. 514/266.2; 544/285

(57) ABSTRACT

Pyrrolidine(thi)one compounds substituted by heterocyclic substituents in the 3-position, their preparation and use in pharmaceutical compositions, in particular as immunomodulators for treatment and/or inhibition of inflammatory and autoimmune diseases and haematological-oncological diseases.

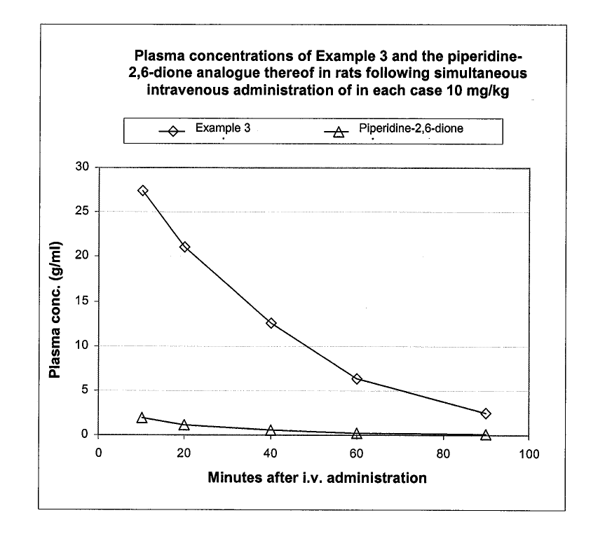

### CROSS REFERENCE TO RELATED APPLICATIONS

This application is a continuation of international patent application no. PCT/EP2006/011440, filed Nov. 29, 2006 designating the United States of America and published in German on Jun. 7, 2007 as WO 2007/062817, the entire disclosure of which is incorporated herein by reference. Priority is claimed based on Federal Republic of Germany patent application no. DE 10 2005 057 912.4, filed Dec. 2, 2005.

### BACKGROUND OF THE INVENTION

The invention relates to pyrrolidine(thi)ones substituted by heterocyclic substituents in the 3-position of the general formula (I)

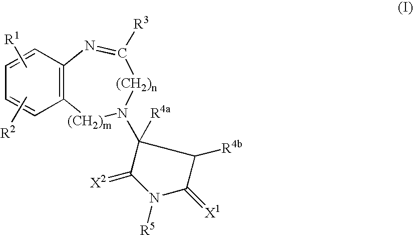

their preparation and their use in medicaments for treatment or inhibition of inflammatory, autoimmune and/or hematologic-oncologic diseases.

Autoimmune diseases arise because of a reactivity of the immune system towards endogenous structures. In this context, the tolerance which is normally present towards endogenous tissue is cancelled out. In addition to antibodies, T lymphocytes and monocytes/macrophages in particular play a decisive role in the pathogenesis of the various autoimmune diseases. Activated monocytes/macrophages secrete a large number of various inflammation-promoting mediators which are responsible directly or indirectly for destruction of the tissue affected by the autoimmune disease. Monocytes/macrophages are activated either in interaction with T lymphocytes or via bacterial products such as lipopolysaccharide (LPS). Interleukin-12 (IL-12) is an inflammation-promoting substance formed by activated monocytes/macrophages.

IL-12 is a heterodimeric molecule which comprises a covalently bonded p35 and p40 chain. It is formed by antigen-presenting cells (monocytes/macrophages, dendritic cells, B lymphocytes) after activation by various microbial products, such as LPS, lipopeptides, bacterial DNA, or in interaction with activated T lymphocytes (Trinchieri 1995. Ann. Rev. Immunol. 13: 251). IL-12 has a central immunoregulatory importance and is responsible for the development of inflammation-promoting TH1 reactivities. If a TH1 immune reaction towards endogenous antigens exists, severe diseases occur, as is clearly documented in numerous animal studies and initial clinical investigations. The pathophysiological importance of IL-12 manifests itself in various animal models for diseases such as rheumatoid arthritis, multiple sclerosis, diabetes mellitus and inflammatory intestinal, skin and mucosa diseases (Trembleau et al. 1995. Immunol. Today 16: 383; Müller et al. 1995. J. Immunol. 155: 4661; Neurath et al. 1995. J. Exp. Med. 182: 1281; Segal et al. 1998. J. Exp. Med. 187: 537; Powrie et al. 1995. Immunity 3: 171; Rudolphi et al. 1996. Eur. J. Immunol. 26: 1156; Bregenholt et al. 1998. Eur. J. Immunol. 28: 379). By administration of IL-12, it was possible to induce the particular disease, or after neutralization of endogenous IL-12 an attenuated course of the disease up to curing of the animals manifested itself. Antibodies against IL-12 are already undergoing clinical trials for treatment of Crohn's disease, psoriasis and multiple sclerosis.

The cytokine IL-10 inhibits synthesis of the inflammation-promoting cytokines TNFα, IL-1, IL-6, IL-8, IL-12 and GM-CSF by human and murine monocytes/macrophages (Fiorentino et al., 1991. J. Immunol. 146: 3444; De Waal Malefyt et al. 1991. J. Exp. Med. 174:1209). By this means an inhibition of the synthesis of IFN-γ by TH1 lymphocytes also indirectly occurs. Interestingly, the formation of IL-10 by monocytes/macrophages occurs with a short time lag with respect to the synthesis of the inflammation-promoting cytokines. Treatment of antigen-presenting cells with IL-10 results in deactivation thereof. Such cells are not capable of activating T lymphocytes to proliferation or to synthesis of IFN-γ. However, these T lymphocytes themselves secrete large amounts of IL-10 and are capable of suppressing inflammation reactions, as it has been possible to demonstrate on the example of an animal model for inflammatory intestinal diseases (Groux et al., 1997. Nature 389: 737). The development of inflammatory skin diseases can also be prevented by IL-10 (Enk et al., 1994. J. Exp. Med. 179: 1397).

Summarizing, it can be said that an excess of IL-12 or a deficiency of IL-10 is the cause of the pathophysiology of a large number of inflammatory/autoimmune diseases. Approaches for re-establishing the equilibrium between inflammation-promoting (IL-12) and inflammation-inhibiting (IL-10) cytokines have therefore a great therapeutic potential in the abovementioned diseases.

IL-12 is moreover also involved in regulation of the survival of cells. Uncontrolled cell growth is regulated inter alia by apoptosis (programmed cell death). It has been demonstrated on T lymphocytes that IL-12 has an anti-apoptotic action and promotes the survival of T cells (Clerici et al. 1994. Proc. Natl. Acad. Sci. USA 91: 11811; Estaquier et al. 1995. J. Exp. Med. 182: 1759). A local over-production of IL-12 can therefore contribute towards the survival of tumour cells. Inhibitors of the formation of IL-12 therefore also have a great therapeutic potential in tumour therapy.

A substance having the immunomodulatory action principle of inhibition of IL-12 and increase in IL-10 is thalidomide. Clinical studies have recently demonstrated the positive influence of thalidomide on the following diseases: erythema nodosum leprosum (Sampaio et al. 1993. J. Infect. Dis. 168: 408), aphthosis (Jacobson et al. 1997. N. Engl. J. Med. 336: 1487), chronic rejection reactions (Vogelsang, et al. 1992. N. Engl. J. Med. 326: 1055), inflammatory intestinal diseases (Ehrenpreis et al. 1999. Gastroenterology 117: 1271; Vasiliauskas et al. 1999. Gastroenterology 117: 1278) and numerous skin diseases (Bernal et al. 1992. Int. J. Derm. 31: 599). Clinical studies are currently also running on therapy for a number of tumour diseases (Rajkumar, 2001. Oncology 15: 867). An activity on multiple myeloma seems certain (Singhal, 1999. N. Engl. J. Med. 341: 1565).

However, thalidomide also induces a number of side effects, including sedation, teratogenicity and neuropathy. Furthermore, the substance is poorly soluble and highly sensitive to hydrolysis.

### SUMMARY OF THE INVENTION

It is therefore an object of the present invention to provide new compounds which exhibit the immunomodulating principle described above.

It has now been found that these requirements imposed on the compounds to be generated are met by certain substituted pyrrolidine(thi)ones.

The invention accordingly provides pyrrolidine(thi)ones substituted by heterocyclic substituents in the 3-position corresponding to formula (I)

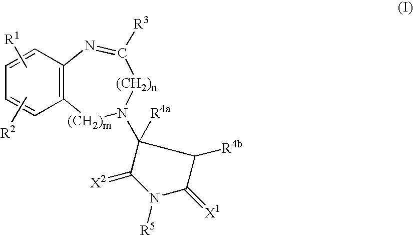

wherein
R¹ and R² independently of one another are chosen from H or any desired radicals,
R³ is chosen from H, aryl, heteroaryl, in each case substituted or unsubstituted, C₁-C₁₀-alkyl, C₂-C₁₀-alkenyl or C₃-C₁₀-alkynyl, in each case branched or unbranched, mono- or polysubstituted or unsubstituted; C₃-C₇-cycloalkyl, saturated or unsaturated, mono- or polysubstituted or unsubstituted, or a corresponding heterocyclic radical in which a C atom in the ring is replaced by S, O or NR^{3′},
   where R³ is chosen from
      H, C₁-C₁₀-alkyl, C₂-C₁₀-alkenyl or C₂-C₁₀-alkynyl, in each case branched or unbranched, mono- or polysubstituted or unsubstituted;
      alkylaryl, saturated or unsaturated, mono- or polysubstituted or unsubstituted; aryl, mono- or polysubstituted or unsubstituted; or
      if C—N single bond is present, represents OH, C₁₋₃-alkoxy or an [O(CO)C₁₋₃-alkyl] group, or together with the C atom represents a carbonyl group,
R^{4a} and/or R^{4b} denote H, F, alkyl, aryl, heteroaryl, in each case substituted or unsubstituted,
R⁵ represents H, aryl, heteroaryl, in each case substituted or unsubstituted, alkyl, a CH₂—OH group or a radical CH₂—NR⁶R⁷, in which R⁶ and R⁷ are identical or different and denote an alkyl group having 1-6 C atoms (straight-chain or branched) or together with the N atom denote a pyrrolidine, piperidine, hexamthyleneimine or morpholine ring,
X¹ and/or X² represent O or S and the remaining X¹ or X² denotes H₂,
n denotes 0 or 1, and
m denotes 1 or 2.

Preferred compounds are those in which
R¹ and R² are identical or different from one another and can be H, Br, Cl, F, I, CF₃, OH, NO₂, NR^{5′}R^{6′}, alkyl, alkoxy, alkylthio, alkylsulfinyl, alkylsulfonyl, aryl, heteroaryl, in each case substituted or unsubstituted, branched or unbranched, or together denote a fused-on benzene ring, wherein the rings are optionally substituted by R¹ and/or R² and R¹ and R² are as defined above, and R^{5′} and R^{6′} are chosen from H, or alkyl or acyl radicals,
R³ represents H, the methyl group or, in the case where a C—N single bond is present, together with the C atom represents a carbonyl group,
R^{4a} and/or R^{4b} denotes H, alkyl, aryl or heteroaryl,
R⁵ represents H, aryl, heteroaryl, in each case substituted or unsubstituted, alkyl, a CH₂—OH group or a radical CH₂—NR⁶R⁷, in which R⁶ and R⁷ are identical or different and denote an alkyl group having 1-6 C atoms (straight-chain or branched) or together with the N atom denote a pyrrolidine, piperidine, hexamthyleneimine or morpholine ring,
X¹ and/or X² represent O or S, and the remaining X¹ or X² denotes H₂,
n denotes 0 or 1, and
m denotes 1 or 2.

Particularly preferred compounds are those having a C═N double bond in which R¹ and R² are identical or different from one another and denote H, Br, Cl, F, CF₃, NO₂, NH₂, C₁₋₃-alkyl, C₁₋₃-alkoxy, or together a fused-on benzene ring, R³ represents H or a methyl group, R^{4a} represents H or a methyl group, R^{4b} represents H or a phenyl group and R⁵ represents H or methyl, X¹ and X² represent O and n=0 and m=1.

Compounds which are in turn preferred are those having a C═N double bond in which R¹ and R² are identical or different from one another and denote H, Cl or F, R³, R^{4a}, R^{4b} and R⁵ denote hydrogen, X¹ and X² represent O and n=0 and m=1.

Further preferred compounds include:
3-(5-chloro-7-fluoro-4H-quinazolin-3-yl)-pyrrolidine-2,5-dione (1),
3-(7-chloro-5-fluoro-4H-quinazolin-3-yl)-pyrrolidine-2,5-dione (2),
3-(7-fluoro-4H-quinazolin-3-yl)-pyrrolidine-2,5-dione and the hydrochloride thereof (3),
3-(5,7-difluoro-4H-quinazolin-3-yl)-pyrrolidine-2,5-dione and the hydrochloride thereof (4),
3-(5,7-dichloro-4H-quinazolin-3-yl)-pyrrolidine-2,5-dione (5),
3-(5-bromo-4H-quinazolin-3-yl)-pyrrolidine-2,5-dione (6),
3-(7-trifluoromethyl-4H-quinazolin-3-yl)pyrrolidine-2,5-dione (7),
3-(5,8-dichloro-4H-quinazolin-3-yl)-pyrrolidine-2,5-dione (8a),
3-(5-methyl-4H-quinazolin-3-yl)-pyrrolidine-2,5-dione (8b),
3-(4H-benzo[g]quinazolin-3-yl)-pyrrolidine-2,5-dione (8c),
3-(6,7-difluoro-4H-quinazolin-3-yl)-pyrrolidine-2,5-dione (8d),
3-(6,8-dichloro-4H-quinazolin-3-yl)-pyrrolidine-2,5-dione (8e),
3-(6-nitro-4H-quinazolin-3-yl)-pyrrolidine-2,5-dione (8f),
3-(7-chloro-4H-quinazolin-3-yl)-pyrrolidine-2,5-dione (8g),
3-(7-nitro-4H-quinazolin-3-yl)-pyrrolidine-2,5-dione (8h),
3-(8-bromo-6-methyl-4H-quinazolin-3-yl)-pyrrolidine-2,5-dione (81),
3-(8-chloro-4H-quinazolin-3-yl)-pyrrolidine-2,5-dione (8j),
3-(8-methoxy-4H-quinazolin-3-yl)-pyrrolidine-2,5-dione (8k),
3-(6-benzyloxy-4H-quinazolin-3-yl)-pyrrolidine-2,5-dione (81),
3-(5,6-dichloro-4H-quinazolin-3-yl)-pyrrolidine-2,5-dione (9a),
3-(7-methoxy-4H-quinazolin-3-yl)-pyrrolidine-2,5-dione (9b),
3-(5-chloro-4H-quinazolin-3-yl)-pyrrolidine-2,5-dione (10a),
3-(6,7-dimethoxy-4H-quinazolin-3-yl)-pyrrolidine-2,5-dione (10b),
3-(6-chloro-4H-quinazolin-3-yl)-pyrrolidine-2,5-dione (10c),
3-(4H-quinazolin-3-yl)-pyrrolidine-2,5-dione (11a),
3-(5-fluoro-4H-quinazolin-3-yl)-pyrrolidine-2,5-dione (11b),
3-(8-trifluoromethyl-4H-quinazolin-3-yl)-pyrrolidine-2,5-dione (II c),
1-methyl-3-(4H-quinazolin-3-yl)-pyrrolidine-2,5-dione (11d),
7-fluoro-1-methyl-3-(4H-quinazolin-3-yl)-pyrrolidine-2,5-dione (11e),
3-(5-methoxy-4H-quinazolin-3-yl)-pyrrolidine-2,5-dione (12a),
3-(5-ethoxy-4H-quinazolin-3-yl)-pyrrolidine-2,5-dione (12b),
3-(5-pentyloxy-4H-quinazolin-3-yl)-pyrrolidine-2,5-dione (12c),
3-(5-benzyloxy-4H-quinazolin-3-yl)-pyrrolidine-2,5-dione (12d),
3-(5-isopropoxy-4H-quinazolin-3-yl)-pyrrolidine-2,5-dione (12e),
3-(5-(2-methoxyethoxy)-4H-quinazolin-3-yl)-pyrrolidine-2,5-dione (12f,
3-(5-ethanesulfonyl-4H-quinazolin-3-yl)-pyrrolidine-2,5-dione (13a),
3-(5-ethanesulfinyl-4H-quinazolin-3-yl)-pyrrolidine-2,5-dione (13c),
3-(5-ethylthio-4H-quinazolin-3-yl)-pyrrolidine-2,5-dione (13b),
3-(5-nitro-4H-quinazolin-3-yl)-pyrrolidine-2,5-dione (14),
1-methyl-3-(2-methyl-4H-quinazolin-3-yl)-pyrrolidine-2,5-dione (15a),
3-(2-methyl-4H-quinazolin-3-yl)-pyrrolidine-2,5-dione (15b),
3-(5,6-dichloro-2-methyl-4H-quinazolin-3-yl)-pyrrolidine-2,5-dione (15c),
3-(5,7-dichloro-2-methyl-4H-quinazolin-3-yl)-pyrrolidine-2,5-dione (15d),
3-(5,7-difluoro-2-methyl-4H-quinazolin-3-yl)-pyrrolidine-2,5-dione and the hydrochloride thereof (15e),
3-(5,8-dichloro-2-methyl-4H-quinazolin-3-yl)-pyrrolidine-2,5-dione (15H),
3-(5-benzyloxy-2-methyl-4H-quinazolin-3-yl)-pyrrolidine-2,5-dione (15g),
3-(5-bromo-2-methyl-4H-quinazolin-3-yl)-pyrrolidine-2,5-dione (15h),
3-(5-chloro-2-methyl-4H-quinazolin-3-yl)-pyrrolidine-2,5-dione (15i),
3-(5-chloro-7-fluoro-2-methyl-4H-quinazolin-3-yl)-pyrrolidine-2,5-dione and the hydrochloride thereof (15j),
3-(5-ethoxy-2-methyl-4H-quinazolin-3-yl)-pyrrolidine-2,5-dione (15k),
3-(5-fluoro-2-methyl-4H-quinazolin-3-yl)-pyrrolidine-2,5-dione (151),
3-(5-isopropoxy-2-methyl-4H-quinazolin-3-yl)-pyrrolidine-2,5-dione (15m),
3-(2,5-dimethyl-4H-quinazolin-3-yl)-pyrrolidine-2,5-dione (15n),
3-(5-methoxy-2-methyl-4H-quinazolin-3-yl)-pyrrolidine-2,5-dione (15o),
3-(2-methyl-5-nitro-4H-quinazolin-3-yl)-pyrrolidine-2,5-dione (15p),
3-(2-methyl-5-pentyloxy-4H-quinazolin-3-yl)-pyrrolidine-2,5-dione (15q),
3-(5-ethylthio-2-methyl-4H-quinazolin-3-yl)-pyrrolidine-2,5-dione (15r),
3-(5-ethanesulfonyl-2-methyl-4H-quinazolin-3-yl)-pyrrolidine-2,5-dione (15s),
3-(2-methyl-4H-benzo[g]quinazolin-3-yl)-pyrrolidine-2,5-dione (15t),
3-(6,7-difluoro-2-methyl-4H-quinazolin-3-yl)-pyrrolidine-2,5-dione (15u),
3-(6,7-dimethoxy-2-methyl-4H-quinazolin-3-yl)-pyrrolidine-2,5-dione (15v),
3-(6,8-dichloro-2-methyl-4H-(quinazolin-3-yl)-pyrrolidine-2,5-dione (15w),
3-(6-benzyloxy-2-methyl-4H-quinazolin-3-yl)-pyrrolidine-2,5-dione (15x),
3-(6-chloro-2-methyl-4H-quinazolin-3-yl)-pyrrolidine-2,5-dione (15y),
3-(2-methyl-6-nitro-4H-quinazolin-3-yl)-pyrrolidine-2,5-dione (15z),
3-(2-methyl-7-trifluoromethyl-4H-quinazolin-3-yl)-pyrrolidine-2,5-dione (15aa),
3-(7-chloro-2-methyl-4H-quinazolin-3-yl)-pyrrolidine-2,5-dione (15bb),
3-(7-fluoro-2-methyl-4H-quinazolin-3-yl)-pyrrolidine-2,5-dione (15 cc),
3-(7-methoxy-2-methyl-4H-quinazolin-3-yl)-pyrrolidine-2,5-dione (15dd),
3-(2-methyl-7-nitro-4H-quinazolin-3-yl)-pyrrolidine-2,5-dione (15ee),
3-(8-bromo-2,6-dimethyl-4H-quinazolin-3-yl)-pyrrolidine-2,5-dione (15ff),
3-(2-methyl-8-trifluoromethyl-4H-quinazolin-3-yl)-pyrrolidine-2,5-dione (15gg),
3-(8-chloro-2-methyl-4H-quinazolin-3-yl)-pyrrolidine-2,5-dione (15hh),
3-(8-methoxy-2-methyl-4H-quinazolin-3-yl)-pyrrolidine-2,5-dione (151i),
3-(5-(2-methoxyethoxy)-2-methyl-4H-quinazolin-3-yl)pyrrolidine-2,5-dione (15jj),
3-(7-amino-4H-quinazolin-3-yl)-pyrrolidine-2,5-dione hydrochloride (16a),
3-(7-amino-2-methyl-4H-quinazolin-3-yl)-pyrrolidine-2,5-dione hydrochloride (16b),
3-(6-amino-4H-quinazolin-3-yl)-pyrrolidine-2,5-dione hydrochloride (16c),
3-(6-amino-2-methyl-4H-quinazolin-3-yl)-pyrrolidine-2,5-dione hydrochloride (16d),
3-(5-amino-4H-quinazolin-3-yl)-pyrrolidine-2,5-dione hydrochloride (16e),
3-(5-amino-2-methyl-4H-quinazolin-3-yl)-pyrrolidine-2,5-dione hydrochloride (16f),
3-(5-hydroxy-4H-quinazolin-3-yl)-pyrrolidine-2,5-dione hydrochloride (17a),
3-(6-hydroxy-2-methyl-4H-quinazolin-3-yl)-pyrrolidine-2,5-dione hydrochloride (17b),
3-(5-hydroxy-2-methyl-4H-quinazolin-3-yl)-pyrrolidine-2,5-dione hydrochloride (17c),
3-(5-hydroxy-2-methyl-4H-quinazolin-3-yl)-pyrrolidine-2,5-dione hydrochloride (17d),
3-(7-fluoro-4H-quinazolin-3-yl)-5-thioxopyrrolidin-2-one hydrobromide (18a),
3-(4H-quinazolin-3-yl)-5-thioxopyrrolidin-2-one hydrobromide (18b),
3-methyl-3-(4H-quinazolin-3-yl)-pyrrolidine-2,5-dione (19a),
3-(5-bromo-4H-quinazolin-3-yl)-3-methyl-pyrrolidine-2,5-dione (19b),
3-(2-oxo-1,4-dihydro-2H-quinazolin-3-yl)-pyrrolidine-2,5-dione (20a),
3-(7-fluoro-2-oxo-1,4-dihydro-2H-quinazolin-3-yl)-pyrrolidine-2,5-dione (20b),
3-(2-oxo-1,4-dihydro-2H-quinazolin-3-yl)-4-phenylpyrrolidine-2,5-dione (20c),
3-(2-thio-1,4-dihydro-2H-quinazolin-3-yl)-pyrrolidine-2,5-dione (20d),
3-(2-(methylthio)-4H-quinazolin-3-yl)pyrrolidine-2,5-dione hydroiodide (21),
3-(2-(dimethylamino)-4-quinazolin-3-yl)pyrrolidine-2,5-dione hydroiodide (22), and
3-(4H-quinazolin-3-yl)-pyrrolidin-2-one (23).

Very particularly preferred compounds of these are:
3-(7-fluoro-4H-quinazolin-3-yl)-pyrrolidine-2,5-dione and the hydrochloride thereof (3), and
3-(5,7-difluoro-4H-quinazolin-3-yl)-pyrrolidine-2,5-dione and the hydrochloride thereof (4).

Radicals in the context of this invention are understood here as meaning replacement of at least one hydrogen by F, Cl, Br, I, CN, CF₃, OCF₃, SR, NO₂, C₁₋₁₀-alkyl, C₂-C₁₀-alkenyl or C₃-C₁₀-alkynyl, in each case branched or unbranched, mono- or polysubstituted or unsubstituted; C₃-C₇-cycloalkyl, saturated or unsaturated, mono- or polysubstituted or unsubstituted, where polysubstituted radicals are to be understood as meaning radicals which are substituted several times either on different or on the same atoms, for example three times on the same C atom as in the case of CF₃ or at different places as in the case of —CH(OH)—CH═CH—CHCl₂,

or a corresponding heterocyclic radical in which a C atom in the ring is replaced by S, O or NR^{5′}, where R⁵ is chosen from
H, C₁-C₁₀-alkyl, C₂-C₁₀-alkenyl or C₂-C₁₀-alkynyl, in each case branched or unbranched, mono- or polysubstituted or unsubstituted;
   OR^{6′}, OC(O)R^{6′}, OC(S)R^{6′}, C(O)R^{6′}, C(O)OR^{6′}, C(S)R^{6′}, C(S)OR^{6′}, SR^{6′}, S(O)R^{6′} or S(O₂)R^{6′}, wherein R^{6′} is chosen from
H, C₁-C₁₀-alkyl, C₂-C₁₀-alkenyl or C₃-C₁₀-alkynyl, in each case branched or unbranched, mono- or polysubstituted or unsubstituted; C₃-C₇-cycloalkyl, saturated or unsaturated, mono- or polysubstituted or unsubstituted, or a corresponding heterocyclic radical in which a C atom in the ring is replaced by S, or NR⁷, where R⁷ is chosen from
   H, C₁-C₁₀-alkyl, C₂-C₁₀-alkenyl or C₃-C₁₀-alkynyl, in each case branched or unbranched, mono- or polysubstituted or unsubstituted;
alkylaryl, saturated or unsaturated, mono- or polysubstituted or unsubstituted; aryl or heteroaryl, in each case mono- or polysubstituted or unsubstituted;
   NR⁸R⁹, C(O)NR⁸R⁹ or S(O₂)NR⁸R⁹, wherein R⁸ and R⁹ independently of one another are chosen from
H, C₁-C₁₈-alkyl, C₂-C₁₈-alkenyl or C₃-C₁₈-alkynyl, in each case branched or unbranched, mono- or polysubstituted or unsubstituted; C₃-C₇-cycloalkyl, saturated or unsaturated, mono- or polysubstituted or unsubstituted, or a corresponding heterocyclic radical in which a C atom in the ring is replaced by S, O or NR¹⁰, where R¹⁰ is chosen from
   H, C₁-C₁₀-alkyl, C₂-C₁₀-alkenyl or C₃-C₁₀-alkynyl, in each case branched or unbranched, mono- or polysubstituted or unsubstituted;
alkylaryl, saturated or unsaturated, mono- or polysubstituted or unsubstituted; aryl or heteroaryl, in each case mono- or polysubstituted or unsubstituted; or
R⁸ and R⁹ together form a C₃-C₇-cycloalkyl, saturated or unsaturated, mono- or polysubstituted or unsubstituted, or a corresponding heterocyclic radical in which a C atom in the ring is replaced by S, O or NR¹⁰, where R¹⁰ is chosen from:
   H, C₁-C₁₀-alkyl, C₂-C₁₀-alkenyl or C₃-C₁₀-alkynyl, in each case branched or unbranched, mono- or polysubstituted or unsubstituted; or
      alkylaryl, aryl or heteroaryl, in each case mono- or polysubstituted or unsubstituted.

In the context of this invention the expression “C₁-C₁₀-alkyl” denotes hydrocarbons having 1 to 10 carbon atoms. Examples which may be mentioned include methyl, ethyl, propyl, isopropyl, n-butane, sec-butyl, tert-butyl, n-pentane, neopentyl, n-hexane, n-heptane, n-octane, n-nonane, n-decane, unsubstituted or mono- or polysubstituted.

In the context of this invention the expression “C₂-C₁₀-alkenyl” or “C₂-C₁₀-alkynyl” denotes hydrocarbons having 2 to 10 carbon atoms. Examples which may be mentioned include ethenyl, propenyl, butenyl, pentenyl, hexenyl, heptenyl, octenyl, unsubstituted or mono- or polysubstituted, and, respectively, ethynyl, propynyl, butynyl, pentynyl, hexynyl, heptynyl, octynyl, unsubstituted or mono- or polysubstituted.

In the context of this invention the expression C₃-C₇-cycloalkyl denotes cyclic hydrocarbons containing 3 to 7 carbon atoms. Examples which may be mentioned include cyclopropyl, cyclobutyl, cyclopentyl, cyclohexyl, cycloheptyl, cyclohexenyl or cycloheptenyl, saturated or unsaturated, unsubstituted or mono- or polysubstituted.

In the context of the invention a “corresponding heterocyclic radical” is understood here as meaning a C₃-C₇-cycloalkyl group in which at least one C atom in the ring is replaced by S, O or N. Examples which may be mentioned include pyrrolidine, pyran, thiolane, piperidine and tetrahydrofuran.

In the context of this invention the expression “aryl” denotes phenyls or naphthyls.

In the context of this invention the expression “alkylaryl” denotes aryls substituted by C₁-C₁₀-alkyls, wherein the expressions aryl and alkyl have the same meaning as above.

In the context of this invention the expression “heteroaryl” denotes 5- or 6-membered aromatic compounds which are optionally provided with a fused-on aryl system and contain one or two hetero atoms selected from the group of nitrogen, oxygen and/or sulfur. Examples which may be mentioned include furan, thiophene, pyrrole, pyridine, pyrimidine, quinoline, isoquinoline, phthalazine or quinazoline.

In connection with “alkyl”, in the context of this invention the term “substituted” is understood as meaning replacement of one or more hydrogen atoms by F, Cl, Br, I, —CN, NH₂, NH-alkyl, NH-aryl, NH-heteroaryl, NH-cycloalkyl, NH-alkyl-aryl, NH-alkyl-heteroaryl, NH-alkyl-OH, N(alkyl)₂, N(alkyl-aryl)₂, N(alkyl-heteroaryl)₂, N(cycloalkyl)₂, N(alkyl-OH)₂, NO₂, SH, S-alkyl, S-aryl, S-heteroaryl, S-alkyl-aryl, S-alkyl-heteroaryl, S-cycloalkyl, S-alkyl-OH, S-alkyl-SH, OH, O-alkyl, O-aryl, O-heteroaryl, O-alkyl-aryl, O-alkyl-heteroaryl, O-cycloalkyl, O-alkyl-OH, CHO, C(═O)C₁₋₆-alkyl, C(═S)C₁₋₆-alkyl, C(═O)aryl, C(═S)aryl, C(═O)C₁₋₆-alkyl-aryl, C(═S)C₁₋₆-alkyl-aryl, C(═O)-heteroaryl, C(═S)-heteroaryl, C(═O)-cycloalkyl, C(═S)-cycloalkyl, CO₂H, CO₂-alkyl, CO₂-alkyl-aryl, C(═O)NH₂, C(═O)NH-alkyl, C(═O)NHaryl, C(═O)NH-cycloalkyl, C(═O)N(alkyl)₂, C(═O)N(alkyl-aryl)₂, C(═O)N(alkyl-heteroaryl)₂, C(═O)N(cycloalkyl)₂, SO-alkyl, SO₂-alkyl, SO₂NH₂, SO₃H, PO(O—C₁₋₆-alkyl)₂, Si(C₁₋₆-alkyl)₃, Si(C₃₋₈-cycloalkyl)₃, Si(CH₂—C₃₋₈-cycloalkyl)₃, Si(phenyl)₃, cycloalkyl, aryl or heteroaryl, wherein polysubstituted groups are to be understood as meaning those groups which are substituted multiple times, e.g. di- or trisubstituted, either on different or on the same atoms, for example trisubstituted on the same C atom as in the case of CF₃ or —CH₂CF₃, or at various places as in the case of —CH(OH)—CH═CH—CHCl₂. The polysubstitution can be by the same or by different substituents. A substituent can optionally also in its turn be substituted; thus, —O-alkyl also includes, inter alia, —O—CH₂—CH₂—O—CH₂—CH₂—OH.

In the context of this invention, with reference to “aryl”, “heteroaryl” and “cycloalkyl”, “mono- or polysubstituted” is understood as meaning mono- or poly-, e.g. di-, tri-tetra- or pentasubstitution of one or more hydrogen atoms of the ring system by F, Cl, Br, I, CN, NH₂, NH-alkyl, NH-aryl, NH-heteroaryl, NH-alkyl-aryl, NH-alkyl-heteroaryl, NH-cycloalkyl, NH-alkyl-OH, N(alkyl)₂, N(alkyl-aryl)₂, N(alkyl-heteroaryl)₂, N(cycloalkyl)₂, N(alkyl-OH)₂, NO₂, SH, S-alkyl, S-cycloalkyl, S-aryl, S-heteroaryl, S-alkyl-aryl, S-alkyl-heteroaryl, S-cycloalkyl, S-alkyl-OH, S-alkyl-SH, OH, O-alkyl, O-cycloalkyl, O-aryl, O-heteroaryl, O-alkyl-aryl, O-alkyl-heteroaryl, O-cycloalkyl, O-alkyl-OH, CHO, C(═O)C₁₋₆-alkyl, C(═S)C₁₋₆-alkyl, C(═O)aryl, C(═S)aryl, C(═O)—C₁₋₆-alkyl-aryl, C(═S)C₁₋₆-alkyl-aryl, C(═O)-heteroaryl, C(═S)-heteroaryl, C(═O)-cycloalkyl, C(═S)-cycloalkyl, CO₂H, CO₂-alkyl, CO₂-alkyl-aryl, C(═O)NH₂, C(═O)NH-alkyl, C(═O)NHaryl, C(═O)NH-cycloalkyl, C(═O)N(alkyl)₂, C(═O)N(alkyl=aryl)₂, C(═O)N(alkyl-heteroaryl)₂, C(═O)N(cycloalkyl)₂, S(O)-alkyl, S(O)-aryl, SO₂-alkyl, SO₂-aryl, SO₂NH₂, SO₃H, CF₃, ═O, ═S; alkyl, cycloalkyl, aryl and/or heteroaryl; on one or optionally different atoms (wherein a substituent can optionally in its turn be substituted). The polysubstitution here is by the same or different substituents.

The compounds according to the invention may be in the form of pure enantiomers or non-racemic enantiomer mixtures, racemates, diastereomers or diastereomer mixtures, in the form both of their free bases and of salts with physiologically acceptable organic or inorganic acids.

The present invention also provides processes for preparing the compounds of the formula (I) according to the invention.

The invention accordingly provides a process for the preparation of compounds of the formula (I) in which X¹ and X² denote oxygen,

wherein 2-aminobenzylamines of the general formula (II)

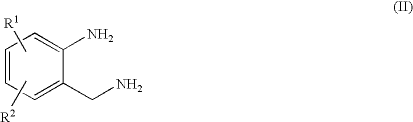

in which R¹ and R² are as defined above, are first reacted with pyrrole-2,5-diones of the general formula (III)

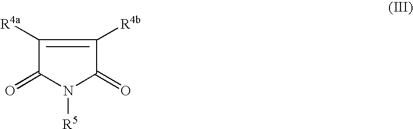

in which R^{4a} and R^{4b} are as defined above, to give amines corresponding to formula (IVa)

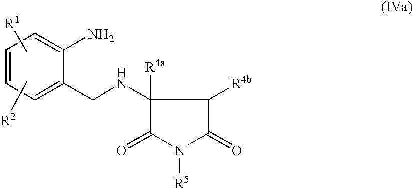

Instead of the pyrrole-2,5-diones, 3-bromo-pyrrolidine-2,5-diones of the general formula (IIIa)

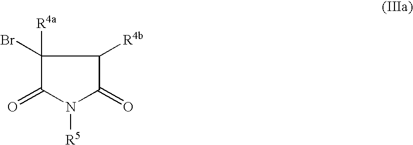

can also be employed.

Compounds of the general formula (IVb) in which at least one O from formula (IVa) has been replaced by S

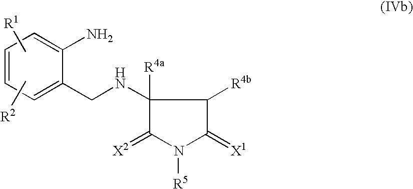

can be obtained from (IVa), preferably after protection of the two nitrogen atoms which are not bonded to R⁵, particularly preferably after protection with benzyloxycarbonyl groups, by exchange of at least one O for X¹ or X² as S with thionation reagents known to persons skilled in the art, and subsequent deprotection. Preferably, this sulfurization is carried out with tetraphosphorus decasulfide, particularly preferably in an inert solvent using ultrasound at temperatures of less than 40° C. If benzyloxycarbonyl protective groups are present, the deprotection can preferably be carried out with hydrogen bromide in acetic acid, the hydrobromides being obtained in this case.

By reaction of (IVa) or (IVb) with compounds of the general formula (V)

R³—C(OR^{y})₃ (V)

in which R³ denotes hydrogen or a methyl group and R^{y} represents a straight-chain or branched C₁-C₄-alkyl group, or with amidine salts of the general formula (VI)

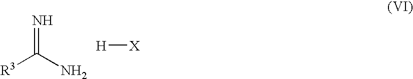

in which R³ is as defined above and X represents the anion of a suitable acid, preferably acetic acid, compounds of the general formula (I) having the corresponding meaning of R³ are then obtained both from (IVa) and from (IVb).

Alternatively, carboxylic acid esters of the general formula (Vb) can also be employed for this.

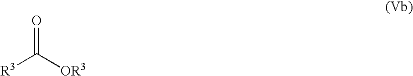

Compounds of the general formula (I) in which R³ together with the C atom represents a germinally substituted C atom and which are to be prepared according to the invention are obtained by reaction of (IVa) or (IVb) with ketones of the general formula (Vc)

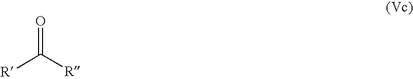

in which R′ and R″ independently of one another represent an alkyl, aryl or heteroaryl group.

Compounds of the general formula (I) in which R³ together with the C atom represents a carbonyl group and which are to be prepared according to the invention are obtained by reaction of (IVa) or (IVb) with Cl units of the general formula (VII)

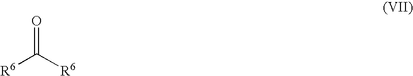

in which R⁶ represents Cl, the imidazol-1-yl group, a C₁-C₄-alkoxy group, a phenyloxy group or a phenyloxy group substituted by the nitro group, chlorine or fluorine or a thiomethyl group, or by reaction of (IVa) or (IVb) with C₁-C₄-alkyl, phenyl or substituted phenyl esters, preferably 4-nitro-, 4-chloro- or 4-fluorophenyl esters, of chloroformic acid.

Compounds of the general formula (I) in which R³ together with the C atom represents a carbonyl group and which are to be prepared according to the invention are also obtained by reaction of (IVa) or (IVb) with a C1 unit of the general formula VIII

C(OR⁷)₄ (VIII)

in which R⁷ represents the methyl or ethyl radical.

Preferably, in the process according to the invention the reaction of the aminobenzylamines of the general formula (II) with the pyrrole-2,5-diones of the general formula (III) is carried out in inert solvents, preferably ethyl acetate, at room temperature.

Likewise preferably, the reaction of the compounds of the general formula (IVa and IVb) with the compounds of the general formula (V) is carried out either without a solvent or in an organic carboxylic acid, preferably acetic acid, in a temperature range of from 10 to 150° C.

The compounds prepared according to the invention can be obtained as pure enantiomers or non-racemic enantiomer mixtures, racemates, diastereomers or diastereomer mixtures, in the form both of their free bases and of salts with physiologically acceptable organic or inorganic acids.

The compounds of the formula (I) are also obtained by first alkylating an amino compound of the general formula (IX)

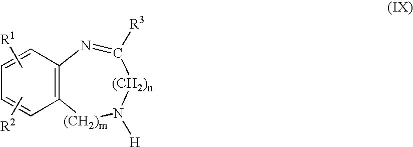

with a pyrrole-2,5-dione of the general formula (III) or a 3-bromopyrrolidine-2,5-dione derivative of the general formula (IIIa)

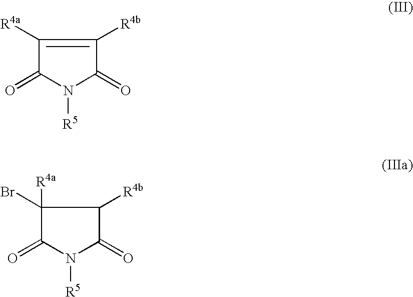

to give a compound of the general formula (Ia)

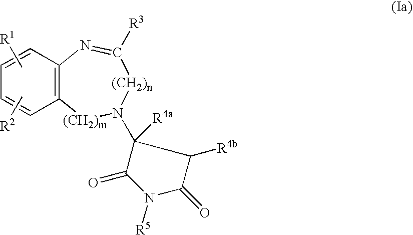

wherein in the compounds (Ia), (II) and (III) the radicals R¹ to R^{4b} are as defined above, and, if R⁵ is not to represent hydrogen, subsequently introducing this radical by reaction with formaldehyde, optionally together with an amine of the general formula HNR⁶R⁷, in which R⁶ and R⁷ are as defined above, and optionally for X¹ and/or X² as S using the sulfurization process described above.

If in the compound of the formula (Ia) the radical R^{4a} represents hydrogen, this can be exchanged for the remaining R^{4a} substituents according to the definition by alkylation or halogenation reactions known per se.

If in the compound of the formula (Ia) R¹ and/or R² represent a nitro group, compounds (Ia) in which R¹ and/or R² denote the amino groups can be prepared therefrom in a manner known per se, e.g. by reduction with catalytically activated hydrogen.

If in the compound of the formula (Ia) R¹ and/or R² represent an amino group, R¹ and/or R² as an acylamino group can be introduced therefrom in a manner known per se by acylation reactions.

If in the compound of the formula (Ia) R¹ and/or R² represent a benzyl-protected group, e.g. benzyloxy, compounds (Ia) in which R¹ and/or R² denote the deprotected group, e.g. OH, can be prepared therefrom in a manner known per se, e.g. by reduction with catalytically activated hydrogen.

Compounds of the formula (I) are also obtained by first alkylating an amino compound of the formula (II) with a 3-bromopyrrolidin-2-one derivative of the formula (X)

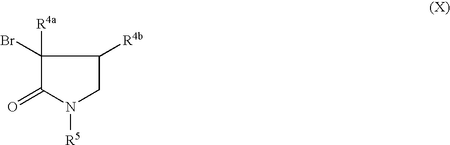

to give a compound of the general formula (Ib)

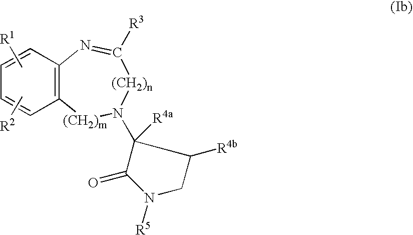

oxidizing this, preferably with m-chloroperbenzoic acid or ruthenium(IV) oxide/sodium periodate, to give a compound of the abovementioned formula (Ia) and optionally introducing other radicals R⁴ and/or the radical R⁵.

2-Aminobenzylamines of the general formula (II) are either commercially available or are prepared in accordance with instructions in the literature or by reduction of corresponding anthranilic acid amides of the general formula (XI)

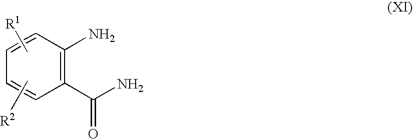

by methods known to persons skilled in the art, e.g. by reduction with borane-dimethyl sulfide complex or with lithium aluminium hydride, in an inert solvent, e.g. tetrahydrofuran, with or without heating.

Anthranilic acid amides of the general formula (X¹) are either commercially available or are prepared in accordance with instructions in the literature or from corresponding anthranilic acids (XII)

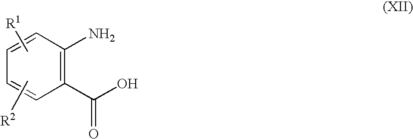

by methods known to the person skilled in the art, e.g. by conversion into anthranilic acid cyanomethyl esters (XIII)

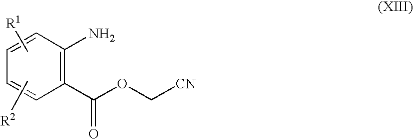

and subsequent reaction with ammonia, e.g. in a dioxane/water mixture, optionally under pressure and at elevated temperature.

Anthranilic acids of the general formula (XII) are either commercially available or are prepared in accordance with instructions in the literature or by methods known to persons skilled in the art, e.g. by reaction of corresponding isatins of the general formula (XIV)

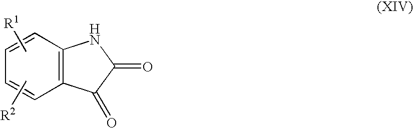

e.g. with alkaline hydrogen peroxide solution, and subsequent addition of acid, e.g. of formic acid.

Isatins of the general formula (XIV) are either commercially available or are prepared in accordance with instructions in the literature or by methods known to persons skilled in the art, e.g. by reaction of corresponding anilines of the general formula (XV)

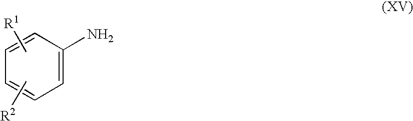

with trichloroacetaldehyde and hydroxylamine hydrochloride to give corresponding 2-hydroxyimino-N-aryl-acetamides (XVI)

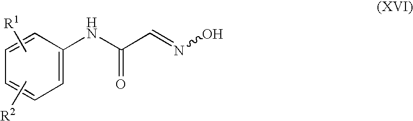

and subsequent acidic cyclization. In the case of unsymmetrically substituted anilines, the formation of regioisomeric isatins can occur here. A separation into the isomers can be carried out either at this stage or in the subsequent syntheses by using purification methods known to the person skilled in the art, e.g. crystallization or chromatography.

Anilines of the general formula (XV) are either commercially available or are prepared in accordance with instructions in the literature or from corresponding carboxylic acids (XVII)

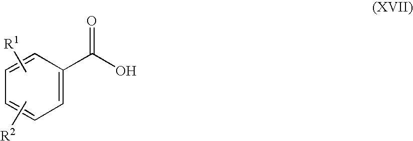

e.g. by reactions which proceed like a Curtius rearrangement. Reagents such as e.g. azidophosphoric acid diphenyl ester in the presence of tert-butanol and a base, e.g. ethyldiisopropylamine, in an inert solvent, e.g. toluene, are advantageously employed here, with heating. The 2-tert-butoxycarbonyl-anilines (XVIII)

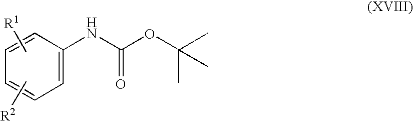

formed in the case where tert-butanol is used can be converted into the anilines (XV) or salts thereof by methods known to the person skilled in the art, e.g. by treatment with hydrogen chloride in dioxane, optionally with the addition of polar solvents, such as e.g. methanol.

A further route for the preparation of the 2-aminobenzylamines of the general formula (II) is the reduction of corresponding 2-aminobenzonitriles of the general formula (XIX)

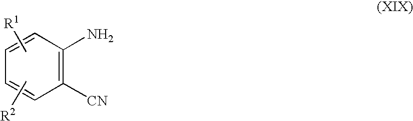

by methods known to persons skilled in the art, e.g. by reduction with borane-dimethyl sulfide complex or with lithium aluminium hydride, in an inert solvent, e.g. tetrahydrofuran, with or without heating.

2-Aminobenzonitriles of the general formula (XIX) are either commercially available or are prepared in accordance with instructions in the literature or from corresponding nitrobenzonitriles of the general formula (XX)

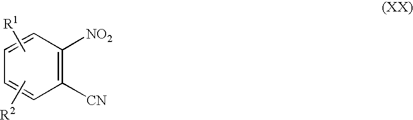

by methods known to the person skilled in the art, e.g. by reduction with hydrogen in the presence of a catalyst, e.g. palladium on active charcoal, in e.g. methanol, or with a metal in a suitable oxidation level, e.g. elemental iron, in e.g. acetic acid, or with tin(II) chloride in a protic solvent.

The conversion of nitrobenzonitriles of the general formula (XX) into anthranilic acid amides of the general formula (XI) can advantageously be carried out in one step by using hydrazine in the presence of Raney nickel.

A further route for the preparation of the 2-aminobenzylamines of the general formula (II) is the aromatic substitution of suitable activating radicals, e.g. the substitution of a nitro group, in dinitrobenzonitriles of the general formula (XXI)

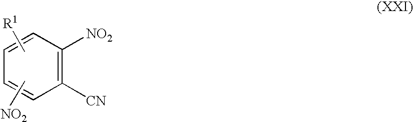

e.g. by alkoxides or thiolates, alkoxy- or, respectively, alkylthio-2-nitrobenzonitriles of the general formulae (XXIIa) and (XXIIb) being obtained.

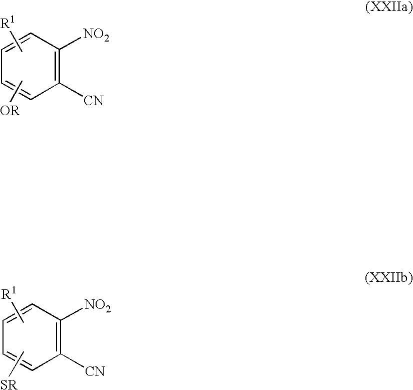

Alkylthio-2-nitrobenzonitriles of the general formula (XXIIb) can be converted with oxidizing agents familiar to the person skilled in the art, e.g. sodium metaperiodate, in the case of n=2 e.g. with addition of chromium(VI) oxide, in e.g. acetonitrile, into compounds of the general formula (XXIIc):

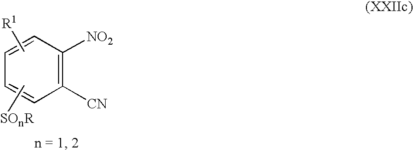

Compounds of the general formulae (XXIIa) to (XXIIc) can be converted into 2-aminobenzylamines of the general formula (II) in the manner described above for 2-nitrobenzonitriles.

Preferred compounds of the formula (I) in which m=1 and n=0 and R³ represents H or OH can also be prepared analogously to the process described in WO 03/053956 A1, by first oxidizing in a manner known per se, e.g. with pyridinium dichromate, a formamide derivative of the general formula (XXIII)

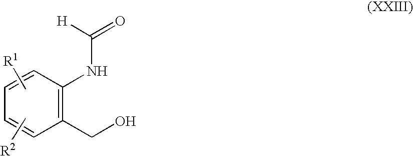

in which R¹ and R² are as defined above and which is accessible by selective N-formylation of the corresponding 2-aminobenzyl alcohol or by selective O-deformylation of the N,O-bisformyl derivative, for example enzymatically with the aid of CAL-B, to give a benzaldehyde or the general formula (XXIV)

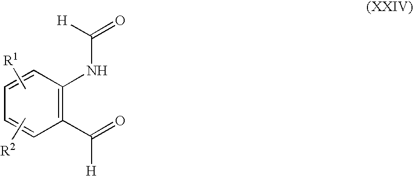

Benzaldehydes of the general formula (XXIV) can be converted by reductive amination with asparagine or corresponding derivatives having a radical R⁴ which is not H using complex borohydrides, such as e.g. sodium borohydride, into compounds of the general formula (XXV).

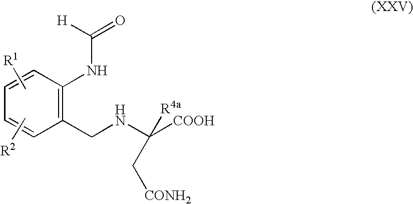

These compounds of the general formula (XXV) can then be cyclized, e.g. with N,N′-carbonyldiimidazole, preferably after prior protection of the amine function by e.g. the benzyloxycarbonyl group, which is subsequently split off again, e.g. with hydrogen bromide in glacial acetic acid, to give succinimides of the general formula (XXVI).

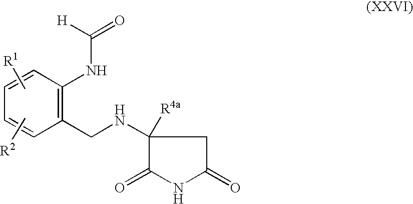

Finally, compounds of the general formula (I) in which R¹, R² and R^{4a} are as defined above, m represents 1, n represents 0, a C═N double bond is present and R³ and R⁵ represent a hydrogen atom which in the case of R⁵ can be exchanged as described above for the remaining substituents according to the definition are obtained from the compounds of the general formula (XXVI) in protic solvents, such as e.g. water, under acid catalysis.

If after reductive amination of the compounds of the general formula (XXIV) the reaction mixture is treated with acids, compounds of the general formula (XXVII) in which R¹, R² and R⁴ are as defined above

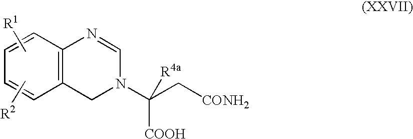

are formed therefrom, and can be converted into compounds of the general formula (I) in which m=1 and n=0, a C═N double bond is present, R¹, R² and R⁴ are as defined above and R³ and R⁵ represent hydrogen, by cyclization, e.g. with acetic anhydride/acetyl chloride.

Thereafter, other radicals R⁴ and/or R⁵ according to the definition are optionally introduced as described above.

Compounds of the formula (I) in which m=2 and the remaining radicals are as defined above can therefore also be obtained in an analogous manner.

The compounds according to the invention have an immunomodulatory activity, and they induce a reduction of IL-12 production in LPS-activated monocytes while simultaneously increasing IL-10 production. Due to this action principle, these compounds have a great therapeutic potential on diseases where excessive IL-12 production and a relative deficiency of IL-10 are held responsible for the pathogenesis, that is to say such compounds are to be used for treatment and/or prophylaxis of inflammatory and autoimmune diseases. Due to the anti-apoptotic action of IL-12, the compounds according to the invention are also suitable for suppression of the formation of IL-12 with haematological-oncological diseases. This distinguishes the compounds of the present invention from known immunomodulators, such as corticosteroids (e.g. dexamethasone), which suppress both the synthesis of IL-12 and that of IL-10 by monocytes.

Surprisingly, the pyrrolidine(thi)ones substituted by heterocyclic substituents in the 3-position of the general formula (I) according to the invention show a good activity compared with the thalidomide analogue in which the glutarimide has been replaced by a succinimide (XXVIII) (Comparison Example 1), which in some cases is comparable to the activity of thalidomide.

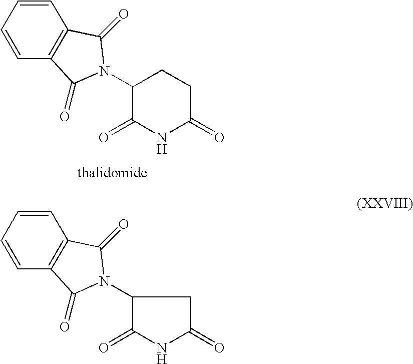

Furthermore, intravenous or oral administrations of certain pyrrolidones substituted by heterocyclic substituents in the 3-position of the general formula (I) surprisingly have the effect of high plasma concentrations in comparison with administration of the same doses of analogous piperidine-2,6-diones. These are to be attributed to a marked reduction in the distribution volumes compared with thalidomide. Since all target cells are either in the blood or supplied intensively with blood, increased plasma concentrations mean improved clinical treatment possibilities.

The advantages over thalidomide which have already been described in WO 03/053956 A1 also manifest themselves in the novel compounds described here, namely a good solubility in water and a lower sensitivity to hydrolysis, as well as improved pharmacokinetic properties.

The diseases of the aforementioned sector include, inter alia, inflammations of the skin (e.g. atopic dermatitis, psoriasis, eczemas, erythema nodosum leprosum), inflammations of the respiratory tract (e.g. bronchitis, pneumonia, bronchial asthma, ARDS (adult respiratory distress syndrome), sarcoidosis, silicosis/fibrosis), inflammations of the gastrointestinal tract (e.g. gastroduodenal ulcers, Crohn's disease, ulcerative colitis), and furthermore diseases such as hepatitis, pancreatitis, appendicitis, peritonitis, nephritis, aphthosis, conjunctivitis, keratitis, uveitis, rhinitis.

The autoimmune diseases include e.g. diseases of the arthritic sector (e.g. rheumatoid arthritis, HLA-B27 associated diseases, rheumatoid spondylitis), and furthermore multiple sclerosis, juvenile-onset diabetes or lupus erythematosus.

Further indications are sepsis, septic shock, bacterial meningitis, mycobacterial infections, opportunistic infections with AIDS, cachexia, transplant rejection reactions, graft-versus-host reactions and chronic cardiac failure, cardiac insufficiency, reperfusion syndrome and atherosclerosis. The indications moreover also include chronic states of pain, fibromyalgia, Sudeck's disease (reflex sympathetic dystrophy (RSD)).

Haematological diseases, such as multiple myeloma, myelodysplastic syndrome and leukaemias, and further oncological diseases, such as e.g. glioblastoma, prostate, kidney cell, mammary, thyroid gland, head and neck, pancreas and colorectal carcinoma and well as melanoma and Kaposi's sarcoma, furthermore belong to the disease syndromes for which the immunomodulators described are to be employed.

Pharmaceutical compositions according to the invention contain, in addition to at least one compound of the general formula I, carrier materials, fillers, solvents, diluents, dyestuffs and/or binders. The choice of auxiliary substances and the amounts to be employed depend on whether the medicament is to be administered orally, rectally, ophthalmically (intravitreally, intracamerally), nasally, topically (including buccally and sublingually), vaginally or parenterally (including subcutaneously, intramuscularly, intravenously, intradermally, intratracheally and epidurally).

Formulations in the form of tablets, chewing tablets, coated tablets, capsules, granules, drops, juices or syrups are suitable for oral administration, and solutions, suspensions, easily reconstitutable dry formulations and sprays are suitable for parenteral, topical and inhalatory administration. Cutaneous administration forms are ointments, gels, creams and pastes. Ophthalmic administration forms include drops, ointments and gels. Compounds according to the invention in a depot in dissolved form, a carrier film or a plaster, optionally with the addition of agents which promote penetration through the skin, are examples of suitable percutaneous administration forms. The compounds according to the invention can be released in a delayed manner from formulation forms for oral or percutaneous use.

The amount of active compound to be administered to patients varies as a function of the weight of the patient, the mode of administration, the indication and the severity of the disease.

### BRIEF DESCRIPTION OF THE DRAWINGS

The accompanying drawing FIGURE is a graph of the plasma concentrations of the compound of the following Example 3 and the piperidine-2,6-analogue thereof in rats following simultaneous intravenous administration of respective 10 mg/kg doses.

### EXAMPLES

The following examples serve to illustrate the present invention in more detail. Silica gel 60 (0.040 to 0.063 mm) from E. Merck, Darmstadt was employed as the stationary phase for the chromatography separations. The mixing ratios of the eluting agents are always stated in volume/volume. The substances were characterized via their melting point, the ¹H-NMR and/or the ¹³C-NMR spectrum. Recording of the spectra at 300 MHz with the Gemini 300 apparatus from Varian. The chemical shifts are stated in ppm (δ scale). Tetramethylsilane (TMS) was used as the internal standard. The example compounds may contain varying small residues of acetic acid which as a rule correspond to between 0 and 3 molar equivalents of acetic acid, based on the particular title compound.

### Comparison Example 1

### 2-(2,5-dioxo-pyrrolidin-3-yl)-isoindole-1,3-dione

The preparation of the title compound can be carried out in accordance with the literature (D. Misiti et al. J. Med. Chem. 1963, 6 464). See also the patent specification GB 1185273.

### Example 1

### 3-(5-chloro-7-fluoro-4H-quinazolin-3-yl)-pyrrolidine-2,5-dione

### Stage 1: (3-chloro-5-fluorophenyl)-carbamic acid tert-butyl ester

A solution of 3-chloro-5-fluorobenzoic acid (2.44 g, 14.0 mmol), diisopropylethylamine (2.8 ml, 2.20 g, 17 mmol) and azidophoshoric acid diphenyl ester (3.7 ml, 4.70 g, 17 mmol) in toluene (10 ml) and tert-butanol (10 ml) was heated under reflux for 16 h. The reaction mixture was then concentrated in vacuo. The residue was taken up in water (20 ml) and the mixture was extracted with ethyl acetate/cyclohexane 1:4 (4×10 ml). The combined organic phases were washed with sodium chloride solution, dried with magnesium sulfate and concentrated. The crude product (4.1 g) was purified by flash chromatography with ethyl acetate/cyclohexane (1:6).

Yield: 2.79 g (81%), white solid.

Melting point: 55-58° C.

### Stage 2: 3-chloro-5-fluoroaniline hydrochloride

A 30% strength solution of hydrogen chloride in 1,4-dioxane (45 ml) was added to the product from stage 1 (27.2 g, 0.11 mmol) in a mixture of 1,4-dioxane (40 ml) and methanol (80 ml) and the mixture was stirred at room temperature for 20 h. The reaction mixture was concentrated in vacuo and diethyl ether (200 ml) was added. The precipitate which had precipitated out was filtered off, washed with diethyl ether and dried in vacuo.

Yield: 18.2 g (91%), white solid.

Melting point: 156-159° C.

### Stage 3: N-(3-chloro-5-fluorophenyl)-2-hydroxyimino-acetamide

A suspension of the product from stage 2 (17.9 g, 98 mmol) in water (10 ml) was added to a solution of chloral hydrate (17.4 g, 105 mmol) and sodium sulfate (113 g) in water (380 ml). A solution of hydroxylamine hydrochloride (21.7 g, 313 mmol) in water (100 ml) was added to this mixture. The reaction mixture was heated under reflux for 40 min. It was then stirred at room temperature for a further 16 h and the product which had precipitated out was filtered off, washed with water and dried over phosphorus pentoxide in vacuo.

Yield: 20.7 g (97%), white solid.

Melting point: 179° C.

### Stage 4: 4-chloro-6-fluoro-1H-indole-2,3-dione (main isomer) and 6-chloro-4-fluoro-1H-indole-2,3-dione (lesser isomer)

The product from stage 3 (20.6 g, 95 mmol) was added to 50-60° C. hot concentrated sulfuric acid (104 ml) in the course of 15 min. The mixture was then heated to 100° C., stirred for 30 min and, after cooling to room temperature, poured over ice (1 kg). The solid which had precipitated out was filtered out, washed neutral with water and dried over phosphorus pentoxide in vacuo.

Yield: 15.5 g (82%) of the mixture of the regioisomers in the ratio of 9:1, yellow solid.

### Main Isomer:

¹H-NMR (DMSO-d₆): 6.72 (0.9H, dd, J=8.8 and 2.0 Hz); 6.77 (0.1H, d, J=2.0 Hz); 7.05 (0.9H, dd, J=9.2 and 2.0 Hz); 7.08 (0.1H, dd, J=9.2 and 4.0 Hz); 11.36 (1H, s).

### Stage 5: 2-amino-6-chloro-4-fluoro-benzoic acid (main isomer) and 2-amino-4-chloro-6-fluoro-benzoic acid (lesser isomer)

First sodium hydroxide (10.7 g, 267 mmol) and then 30% hydrogen peroxide (10.7 ml) in water (96 ml) were added to a suspension of the product mixture from stage 4 (8.00 g, 40 mmol) in water (200 ml) at 0° C. The mixture was stirred at room temperature overnight and then brought to pH 3.3 with formic acid (approx. 20 ml, severe foaming). The title compound which had precipitated out was filtered off and dried over phosphorus pentoxide in vacuo. The aqueous solution was extracted with ethyl acetate (2×150 ml). The pH of the aqueous phase was corrected to 3.3 and extraction was carried out again with ethyl acetate (150 ml). The combined organic phases were dried with sodium sulfate and concentrated and the residue was dried in vacuo.

Yield: 7.30 g (95%) of the mixture of the regioisomers in the ratio of 9:1, brown solid.

### Main Isomer:

¹³C-NMR (DMSO-d₆): 100.2 (d, J=24 Hz); 104.5 (d, J=26 Hz); 112.0; 133.7 (d, J=15 Hz); 151.0 (d, J=13 Hz); 162.9 (d, J=244 Hz); 167.1.

### Stage 6: 2-amino-6-chloro-4-fluoro-benzoic acid cyanomethyl ester (main isomer) and 2-Amino-4-chloro-6-fluoro-benzoic acid cyanomethyl ester (lesser isomer)

Triethylamine (7.6 ml, 55 mmol) and a solution of chloroacetonitrile (3.9 ml, 60 mmol) in acetone (25 ml) were added to a solution of the product mixture from stage 5 (7.00 g, 37 mmol) in acetone (128 ml) and the mixture was stirred at room temperature for 76 h. The mixture was then filtered and the filtrate was concentrated in vacuo. The residue (12.8 g) was dissolved in ethyl acetate, the solution was washed with water and the organic phase was dried with sodium sulfate and concentrated in vacuo.

Yield: 6.00 g (70%) of the mixture of the regioisomers in the ratio of 9:1, brown oil.

### Main Isomer:

¹H-NMR (DMSO-d₆): 5.17 (2H, s); 6.37 (2H, s); 6.40-6.60 (2H, m).

### Stage 7: 2-amino-6-chloro-4-fluoro-benzamide (main isomer) and 2-amino-4-chloro-6-fluoro-benzamide (lesser isomer)

25% aqueous ammonia (7 ml) was added to a solution of the crude product mixture from stage 6 (1.80 g) in 1,4-dioxane (3 ml) and the mixture was stirred in a steel autoclave at 100° C. overnight. After cooling of the reaction solution, this was concentrated in vacuo. The crude product (1 g) was purified by flash chromatography with ethyl acetate/cyclohexane (1:1).

### Lesser Isomer:

Yield: 55 mg (5%), yellow solid.

Melting point: 142-145° C. (decomposition)

¹H-NMR (DMSO-d₆): 6.41 (2H, s); 6.45 (1H, dd, J=10.8 and 2.0 Hz); 6.59 (1H, t, J=2.0 Hz); 7.55 (1H, s); 7.59 (1H, s).

¹³C-NMR (DMSO-d₆): 102.0 (d, J=28 Hz); 104.8 (d, J=18 Hz); 110.7; 135.0 (d, J=16 Hz); 151.7 (d, J=8 Hz); 160.8 (d, J=245 Hz); 165.7.

### Main Isomer:

Yield: 500 mg (47%), white solid.

Melting point: 101-104° C.

¹H-NMR (DMSO-d₆): 5.56 (2H, m); 6.40-6.50 (2H, m); 7.60 (1H, s); 7.82 (1H, s).

¹³C-NMR (DMSO-d₆): 99.6 (d, J=23 Hz); 103.1 (d, J=26 Hz); 118.3; 131.1 (d, J=14 Hz); 148.7 (d, J=13 Hz); 162.1 (d, J=244 Hz); 166.9.

### Stage 8: 2-aminomethyl-3-chloro-5-fluoro-phenylamine

2 M borane-dimethyl sulfide complex (40 ml, 80 mmol) in tetrahydrofuran was added to a solution of the main isomer from stage 7 (4.8 g, 25 mmol) in anhydrous tetrahydrofuran (85 ml) and the mixture was heated under reflux for 6 h. Water (6.5 ml) was cautiously added to the cooled reaction solution and the mixture was then concentrated in vacuo. The residue was repeatedly (3×) dissolved in methanol and concentrated again each time. The crude product (9 g) was purified by flash chromatography with chloroform/methanol (4:1) and 1% triethylamine.

Yield: 2.97 g (68%), yellowish solid.

Melting point: 71-74° C.

### Stage 9: 3-(2-amino-6-chloro-4-fluoro-benzylamino)-pyrrolidine-2,5-dione

A solution of the product from stage 8 (600 mg, 3.4 mmol) and maleimide (500 mg, 5.1 mmol) in ethyl acetate (9 ml) was stirred at room temperature for 22 h. The product which had precipitated out was filtered off and dried in vacuo.

Yield: 820 mg (88%), white solid.

Melting point: 143-144° C.

### Stage 10: 3-(5-chloro-7-fluoro-4H-quinazolin-3-yl)-pyrrolidine-2,5-dione

A solution of the product from stage 9 (308 mg, 1.1 mmol) and orthoformic acid triethyl ester (326 mg, 361 μl, 2.2 mmol) in ethyl acetate (10 ml) was stirred at room temperature for 6 h. The reaction mixture was concentrated in vacuo, toluene was added to the residue and the mixture was concentrated again.

Yield: 400 mg (100%) of the title compound, white solid which contains 1 molar equivalent of acetic acid.

Melting point: 203-204° C.

### Example 2

### 3-(7-chloro-5-fluoro-4H-quinazolin-3-yl)-pyrrolidine-2,5-dione

By replacing the main isomer used in Example 1, stage 8 by the lesser isomer and using the procedure described in stages 8 to 10, the title compound was obtained in the form of a white solid.

Melting point: 179-181° C.

### Example 3

### 3-(7-fluoro-4H-quinazolin-3-yl)-pyrrolidine-2,5-dione

### Stage 1: 2-amino-4-fluorobenzoic acid cyanomethyl ester

First triethylamine (5.03 ml, 36.1 mmol), then a solution of chloroacetonitrile (2.44 ml, 38.7 mmol) in acetone (15 ml) were added to a solution of 2-amino-4-fluorobenzoic acid (3.93 g, 25.3 mmol) in acetone (90 ml) and the mixture was subsequently stirred at room temperature for 2 d. The reaction mixture was filtered, the solid residue was washed with acetone and the filtrate was concentrated in vacuo. The residue was dissolved in ethyl acetate and washed with half-saturated sodium chloride solution. The organic phase was dried with sodium sulfate and concentrated in vacuo.

Yield: 4.22 g (86%), white solid.

Melting point: 69-70° C.

### Stage 2: 2-amino-4-fluorobenzamide

33% aqueous ammonia solution (40 ml) was added to a solution of the product from stage 1 (3.50 g, 18 mmol) in 1,4-dioxane (10 ml) and the mixture was stirred in a closed 80 ml steel autoclave at a bath temperature of 100° C. for 20 h. The reaction solution, cooled to room temperature, was then concentrated in vacuo and the residue was purified by flash chromatography with chloroform/methanol (95:5).

Yield: 2.45 g (88%), white solid.

Melting point: 118-123° C.

¹H-NMR (DMSO-d₆): 6.27 (1H, dt, J=8.6 and 3.1 Hz); 6.43 (1H, dd, J=11.7 and 3.1 Hz); 6.87 (2H, b s); 7.04 (1H br s); 7.59 (1H, dd J=8.6 and 6.3 Hz); 7.67 (1H br s).

### Stage 3: 2-aminomethyl-5-fluorophenylamine

2 M borane-dimethyl sulfide complex (40 ml, 80 mmol) in tetrahydrofuran was added to a solution of the product from stage 2 (2.45 g, 16 mmol) in anhydrous tetrahydrofuran (50 ml) and the mixture was heated under reflux for 9 hours. Water (4 ml) was cautiously added to the cooled reaction solution and the mixture was then concentrated in vacuo. Toluene (2×) was added to the residue and the mixture was concentrated again each time. The residue was then repeatedly dissolved in methanol (3×) and concentrated each time. The crude product was purified by flash chromatography with chloroform/methanol (7:3) and 1% triethylamine.

Yield: 1.37 g (61%), white solid.

Melting point: 49-51° C.

### Stage 4: 3-(2-amino-4-fluorobenzylamino)-pyrrolidine-2,5-dione

A solution of the product from stage 3 (280 mg, 2 mmol) and maleimide (194 mg, 2 mmol) in ethyl acetate (5 ml) was stirred at room temperature for 20 h. The mixture was then stirred at 50° C. for 3 h. Since the reaction was not complete, further maleimide (49 mg, 0.5 mmol) was added and the mixture was stirred again at 50° C. for 18 hours. The reaction mixture was concentrated in vacuo and the residue was purified by flash chromatography with chloroform/methanol (95:5).

Yield: 358 mg (75%).

¹H-NMR (DMSO-d₆): 2.42 (1H, dd, J=17.6 and 5.9 Hz); 2.64 (1H, br s); 2.75 (1H, dd, J=17.6 and 8.8 Hz); 3.57-3.74 (3H, m); 5.47 (2H, s); 6.24 (1H, dt, J=8.8 and 2.9 Hz); 6.37 (1H, dd, J=11.7 and 2.0 Hz); 6.97 (1H, dd, J=7.8 and 7.8 Hz); 11.13 (1H, s).

### Stage 5: 3-(7-fluoro-4H-quinazolin-3-yl)-pyrrolidine-2,5-dione

A solution of the product from stage 4 (311 mg, 1.3 mmol) in acetic acid (10 ml) and orthoformic acid triethyl ester (428 μl, 2.6 mmol) was stirred at room temperature for 24 hours. The reaction mixture was concentrated in vacuo, the residue was taken up in toluene, the mixture was concentrated again and the residue was purified by flash chromatography with ethyl acetate/methanol (4:1).

Yield: 243 mg (76%), yellowish solid.

Melting point: 191° C.

### Stage 6: 3-(7-fluoro-4H-quinazolin-3-yl)-pyrrolidine-2,5-dione hydrochloride

Water (0.02 ml) and then chlorotrimethylsilane (0.12 ml) were added to a solution of the product from stage 5 (230 mg, 0.93 mmol) in butan-2-one (3 ml), while stirring, and the mixture was stirred at room temperature for 30 minutes. The solid formed was filtered off with suction, washed with diethyl ether and dried at 70° C. in a drying cabinet for 15 hours.

Yield: 258 mg (98%), beige-colored solid.

Decomposition from 214° C.

### Example 4

### 3-(5,7-difluoro-4H-quinazolin-3-yl)-pyrrolidine-2,5-dione

### Stage 1: N-(3,5-difluorophenyl)-2-hydroxyiminoacetamide

A suspension of 3,5-difluoroaniline (15.0 g, 116 mmol) in concentrated hydrochloric acid (10 ml) was added to a solution of chloral hydrate (20.5 g, 124 mmol) and sodium sulfate (133.8 g) in water (450 ml). A solution of hydroxylamine hydrochloride (25.7 g, 370 mmol) in water (120 ml) was added to this mixture. The mixture was heated under reflux for 30 min, during which a clear solution formed, from which the solid then precipitated out. The mixture was then stirred at room temperature for a further 3.5 h and the product which had precipitated out was filtered out, washed with a large amount of water and dried over phosphorus pentoxide in a desiccator.

Yield: 21.1 g (91%), white solid.

Melting point: 190-192° C.

### Stage 2: 4,6-difluoro-1H-indole-2,3-dione

The product from stage 1 (21.1 g, 105 mmol) was added to 50-60° C. hot concentrated sulfuric acid (115 ml) in the course of 15 min. The mixture was then heated to 100° C., stirred for 30 min and, after cooling to room temperature, poured slowly on to ice (1.2 kg). The solid which had precipitated out was filtered off, washed neutral with water and dried over phosphorus pentoxide in a desiccator.

Yield: 18.4 g (95%), yellow solid.

¹H-NMR (DMSO-d6): 6.60 (1H, dd, J=8.8 and 2.0 Hz); 6.84 (1H, dt, J=9.8 and 2.0 Hz); 11.20 (1H, br s).

### Stage 3: 2-amino-4,6-difluorobenzoic acid

First sodium hydroxide (6.70 g, 167 mmol) and then 30% hydrogen peroxide (6.7 ml) in water (60 ml) were added to a suspension of the product from stage 2 (4.58 g, 25 mmol) in water (125 ml) at 0° C. The mixture was stirred at room temperature for 20 h and then brought to pH 3 with formic acid (approx. 11 ml, severe foaming), during which the title compound precipitated out. The mixture was then filtered and product was dried over phosphorus pentoxide in a desiccator.

Yield: 3.72 g (86%), white solid.

Melting point: 198-202° C.

### Stage 4: 2-amino-4,6-difluorobenzoic acid cyanomethyl ester

First triethylamine (580 mg, 1.2 ml, 8.7 mmol) and then a solution of chloroacetonitrile (0.6 ml, 9.5 mmol) in acetone (4 ml) were added to a solution of the product from stage 3 (1.00 g, 5.8 mmol) in acetone (20 ml) and the mixture was stirred at room temperature overnight. The mixture was then filtered, the filtrate was concentrated in vacuo, the residue was dissolved in ethyl acetate, the undissolved solid was filtered out and the filtrate was concentrated again. The residue was purified by flash chromatography with cyclohexane/ethyl acetate (4:1).

Yield: 1.01 g (82%), white solid.

Melting point: 71-72° C.

### Stage 5: 2-amino-4,6-difluorobenzamide

25% aqueous ammonia was added to a solution of the product from stage 4 (6.88 g, 32.5 mmol) in 1,4-dioxane (10 ml) and the mixture was stirred in a steel autoclave at 100° C. over the weekend. After cooling, the reaction solution was concentrated in vacuo. A second reaction batch was treated exactly the same in parallel. The crude products were combined and purified together by flash chromatography with chloroform/methanol (95:5).

Yield: 9.04 g (81%), white solid.

Melting point: 113-114° C.

### Stage 6: 2-aminomethyl-3,5-difluorophenylamine

2 M borane-dimethyl sulfide complex (46.5 ml, 93 mmol) in tetrahydrofuran was added to a solution of the product from stage 5 (5.00 g, 29 mmol) in anhydrous tetrahydrofuran (100 ml) and the mixture was heated under reflux for 8 h. Water (7.5 ml) was cautiously added to the cooled reaction solution and the mixture was then concentrated in vacuo. Toluene (2×) was added to the residue and the mixture was concentrated again each time. The residue was then repeatedly dissolved in methanol (3×) and the solution was concentrated each time. The crude product was purified by flash chromatography with chloroform/methanol (7:3) and 1% triethylamine.

Yield: 3.16 g (69%), brownish oil.

¹H-NMR (DMSO-d₆): 1.73 (2H, very br s); 3.65 (2H, s); 5.87 (2H; br s); 6.10-6.30 (2H, m).

### Stage 7: 3-(2-amino-4,6-difluorobenzylamino)-pyrrolidine-2,5-dione

A solution of the product from stage 6 (1.00 g, 6.32 mmol) and maleimide (776 mg, 8 mmol) in ethyl acetate (15 ml) was stirred at 50° C. for 70 h. The product which had precipitated out was then filtered off and dried.

Yield: 1.20 g (75%), colorless solid.

Melting point: 178° C.

### Stage 8: 3-(5,7-difluoro-4H-quinazolin-3-yl)-pyrrolidine-2,5-dione

A solution of the product from stage 7 (300 mg, 1.18 mmol) in acetic acid (10 ml) and orthoformic acid triethyl ester (351 mg 390 μl, 2.37 mmol) was stirred at room temperature for 24 h. Since the reaction was not yet complete, the mixture was stirred at 50° C. for a further 3.5 h, further orthoformic acid triethyl ester (390 μl) was then added and the mixture was stirred at room temperature for 2 hours. The reaction mixture was concentrated in vacuo, the residue was taken up in toluene, the mixture was concentrated again and the crude product was purified by flash chromatography with chloroform/methanol (9:1).

Yield: 247 mg (75%), white solid.

Melting point: 198° C.

### Stage 9: 3-(5,7-difluoro-4H-quinazolin-3-yl)-pyrrolidine-2,5-dione hydrochloride

Water (0.02 ml) and then chlorotrimethylsilane (0.11 ml) were added to a suspension of the product from stage 8 (220 mg, 0.84 mmol) in butan-2-one (3 ml), while stirring, and the mixture was stirred in an ice bath at 0° C. for 30 minutes. The solid formed was filtered off with suction, washed with diethyl ether and dried at 70° C. in a drying cabinet for 15 hours.

Yield: 245 mg (98%), white solid.

Decomposition from 290° C.

### Example 5

### 3-(5,7-dichloro-4H-quinazolin-3-yl)-pyrrolidine-2,5-dione

By replacing the aniline used in Example 4, stage 1 by the corresponding 3,5-dichloro isomer and using the procedure described in stages 1 to 8, the title compound was obtained in the form of a yellowish solid.

Melting point: 215-217° C.

### Example 6

### 3-(5-bromo-4H-quinazolin-3-yl)-pyrrolidine-2,5-dione

By replacing the 1-H-indole-2,3-dione used in Example 4, stage 2 with the corresponding 4-bromo isomer and using the procedure described in stages 2 to 8, the title compound was obtained in the form of a yellow solid.

Melting point: 214-217° C.

### Example 7

### 3-(7-trifluoromethyl-4H-quinazolin-3-yl)-pyrrolidine-2,5-dione

By replacing the 2-aminobenzoic acid used in Example 4, stage 3 with the corresponding starting material and using the procedure described in stages 3 to 8, the title compound was obtained.

Melting point: 213-215° C.

The 2-aminobenzoic acid used as a starting material in this example could be prepared in one step from the corresponding 2-nitrobenzoic acid: 2-amino-4-trifluoromethylbenzoic acid

10% palladium on active charcoal (1.00 g) was added to a solution of 2-nitro-4-trifluoromethylbenzoic acid (10.0 g, 42.6 mmol) in methanol (20 ml) and hydrogenation was carried out at room temperature under a hydrogen atmosphere of 3 bar until 128 mmol of hydrogen had been taken up. The mixture was filtered over silica gel and the filtrate was concentrated in vacuo.

Yield: 8.48 g (97%), white solid.

Melting point: 174-178° C.

### Example 8

By replacing the 2-aminobenzoic acid used in Example 4, stage 3 with the corresponding starting material and using the procedure described in stages 3 to 8, the following compounds were obtained:

### a) 3-(5,8-dichloro-4H-quinazolin-3-yl)-pyrrolidine-2,5-dione

¹H-NMR (DMSO-d₆): 1.91 (3H, s, HOAc); 2.86 (1H, dd, J=18.6 and 9.8 Hz); 3.11 (1H, dd, J=18.6 and 5.9 Hz); 4.14 (1H, d, J=14.7 Hz); 4.67 (1H, d, J=15.7 Hz); 4.94 (1H, dd, J=8.8 and 6.8 Hz); 7.06 (1H, d, J=8.8 Hz); 7.23-7.34 (2H, s and d overlapped); 11.65 (1H, br s); 11.97 (1H, br s, HOAc).

### b) 3-(5-methyl-4H-quinazolin-3-yl)-pyrrolidine-2,5-dione

Melting point: 221-224° C.

### c) 3-(4H-benzo[q]quinazolin-3-yl)-pyrrolidine-2,5-dione

Melting point: 100-102° C.

### d) 3-(6,7-difluoro-4H-quinazolin-3-yl)-pyrrolidine-2,5-dione

### e) 3-(6,8-dichloro-4H-quinazolin-3-yl)-pyrrolidine-2,5-dione

Melting point: 230-232° C.

### f) 3-(6-nitro-4H-quinazolin-3-yl)-pyrrolidine-2,5-dione

Melting point: 168-172° C.

### g) 3-(7-chloro-4H-quinazolin-3-yl)-pyrrolidine-2,5-dione

Melting point: 194° C.

### h) 3-(7-nitro-4H-quinazolin-3-yl)-pyrrolidine-2,5-dione

Melting point: 212-225° C.

### i) 3-(8-bromo-6-methyl-4H-quinazolin-3-yl)-pyrrolidine-2,5-dione

Melting point: 240-241° C.

### j) 3-(8-chloro-4H-quinazolin-3-yl)-pyrrolidine-2,5-dione

Melting point: 194° C.

### k) 3-(8-methoxy-4H-quinazolin-3-yl)-pyrrolidine-2,5-dione

Melting point: 195-200° C.

### l) 3-(6-benzyloxy-4H-quinazolin-3-yl)-pyrrolidine-2,5-dione

Melting point: 171-173° C.

To prepare of the 2-amino-5-benzyloxybenzoic acid employed, the procedure described in the literature (A. Witt, J. Bergman J. Org. Chem. 2001, 66, 2784) was followed with the exception of the last step. Surprisingly, in the last step it was found that during the treatment with hydrochloric acid, not only were the methyl ester and the N-acetyl group hydrolyzed, as necessary, but the benzyl group was also removed. The hydrolysis of the methyl ester was therefore carried out by slower heating under reflux in the presence of sodium hydroxide in a water/dioxane mixture as follows:

Sodium hydroxide (4.64 g, 116 mmol) was added to a suspension of 2-acetylamino-5-benzyloxybenzoic acid methyl ester (13.9 g, 46.4 mmol) in water (120 ml) and 1,4-dioxane (120 ml). The mixture was heated under reflux for 5 hours, during which a solution formed. Stirring was then continued at 80° C. overnight, the mixture was concentrated in vacuo, the residue was taken up in water and the solution was adjusted to pH 3-4 with formic acid. The solid which had precipitated out was filtered out and dried. The resulting mixture was dissolved again in water (250 ml), sodium hydroxide (4.64 g, 116 mmol) was added and the mixture was stirred under reflux for 8 hours and at 80° C. overnight. The reaction mixture was cooled and the solution was adjusted to pH 3-4 with formic acid. The product which had precipitated out was filtered out and dried over phosphorus pentoxide in vacuo.

Yield: 10.2 g (90%) of 2-amino-5-benzyloxybenzoic acid, grey solid.

1H-NMR (DMSO-d₆): 4.97 (2H, s); 6.70 (1H, d, J=9.8 Hz); 7.01 (1H, dd, J=8.8 and 2.9 Hz); 7.28-7.43 (6H, m); 8.40 (3H, very br s).

### Example 9

By replacing the 2-aminobenzamide used in Example 4, stage 5 by the corresponding starting material and using the procedure described in stages 5 to 8, the following compounds were obtained:

### a) 3-(5,6-dichloro-4H-quinazolin-3-yl)-pyrrolidine-2,5-dione

Melting point: 174-178° C.

The 2-aminobenzamide used as a starting material in this example could be prepared in one step from the corresponding 2-nitrobenzonitrile:

### 6-amino-2,3-dichlorobenzamide

85% strength hydrazine hydrate (0.6 ml, 40 mmol) was added to a solution of 2,3-dichloro-6-nitrobenzonitrile (2.17 g, 10 mmol) in ethanol (45 ml) at 45° C. Aqueous 50% Raney nickel suspension was added in portions to this solution until no further evolution of gas was to be observed. The mixture was then boiled under reflux for 2.5 hours. After cooling to room temperature, the reaction mixture was filtered, the residue on the filter was washed with hot ethanol and the filtrate was concentrated in vacuo. The residue was taken up repeatedly in ethanol (2×), concentrated in vacuo each time and then purified by flash chromatography with chloroform/methanol (95:5).

Yield: 1.48 g (72%), white solid.

Melting point: 110-112° C.

### b) 3-(7-methoxy-4H-quinazolin-3-yl)-pyrrolidine-2,5-dione

Melting point: 174-184° C.

The 2-aminobenzamide used in this example could be prepared from the corresponding 2-nitrobenzonitrile analogously to the preceding example:

### 2-amino-4-methoxybenzamide

Melting point: 152-155° C.

### Example 10

By replacing the 2-aminobenzylamine used in Example 4, stage 6 with the corresponding starting material and using the procedure described in stages 6 to 8, the following compounds were obtained:

### a) 3-(5-chloro-4H-quinazolin-3-yl)-pyrrolidine-2,5-dione

Melting point: 218° C.

The 2-aminobenzylamine used as a starting material in this example could be prepared in one step from the corresponding 2-aminobenzonitrile:

A solution of 2-amino-6-chloro-benzonitrile (19.0 g, 125 mmol) in anhydrous tetrahydrofuran (150 ml) was added dropwise to 10.0 g (253 mmol) of lithium aluminium hydride in anhydrous tetrahydrofuran (520 ml) at 20-30° C. and the mixture was then stirred under reflux for 1 h. Excess lithium aluminium hydride was cautiously destroyed with 125 ml of THF/water (2:1) at 0-10° C. The suspension obtained was filtered with suction over kieselguhr and washed with tetrahydrofuran. The filtrate was concentrated in vacuo. The residue was purified by column chromatography with methylene chloride/methanol (4:1).

Yield: 17.3 (88.4%), yellow solid.

¹H-NMR (DMSO-d₆, 300 MHz): 1.76 (2H, br s); 3.81 (2H, s); 5.52 (2H, br s); 6.59 (2H, dd, J=7.9 and 7.9 Hz); 6.91 (1H, t, J=7.9 Hz).

### b) 3-(6,7-dimethoxy-4H-quinazolin-3-yl)-pyrrolidine-2,5-dione

Melting point: 189-200° C.

The 2-aminobenzylamine used as a starting material in this example could be prepared in one step from the corresponding 2-aminobenzonitrile:

### 2-aminomethyl-4,5-dimethoxyphenylamine

A solution of 2-amino-4,5-dimethoxybenzonitrile (2.00 g, 11.2 mmol) in tetrahydrofuran (50 ml) and 2 M borane-dimethyl sulfide complex in tetrahydrofuran (28 ml, 56 mmol) was boiled under reflux for 7 hours. Water (5 ml) was cautiously added to the cooled reaction solution and the mixture was then concentrated in vacuo. The residue was taken up several times in toluene and then in methanol and concentrated again each time. The residue was purified by flash chromatography with chloroform/methanol (9:1 and 1% triethylamine).

Yield: 1.51 g (74%), brownish solid.

Melting point: 86-87° C.

### c) 3-(6-chloro-4H-quinazolin-3-yl)-pyrrolidine-2,5-dione

Melting point: 166° C.

The 2-aminobenzylamine used as a starting material in this example could be prepared in one step from the corresponding 2-aminobenzonitrile analogously to the starting material in Example 10b:

### 2-aminomethyl-5-chlorophenylamine

¹H-NMR (DMSO-d₆): 1.82 (2H, s); 3.58 (2H, s); 5.21 (2H, br s); 6.57 (1H, d, J=8.8 Hz); 6.92 (1H, dd, J=7.8 and 2.0 Hz); 7.07 (1H, d, J=2.0 Hz).

### Example 11

By replacing the 2-aminobenzylamine used in Example 4, stage 6 with the corresponding starting material and using the procedure described in stages 6 to 8, the following compounds were obtained:

### a) 3-(4H-quinazolin-3-yl)-pyrrolidine-2,5-dione

Melting point: 168-174° C.

1H-NMR (DMSO-d6): 2.87 (1H, dd, J=18.6 and 8.8 Hz); 3.00 (1H, dd, J=18.6 and 5.6 Hz); 4.13 (1H, d, J=13.7 Hz); 4.61 (1H, d, J=13.7 Hz); 4.84 (1H, dd, J=8.8 and 5.9 Hz); 6.90 (2H, dd, J=8.8 and 8.8 Hz); 6.98 (1H, dd, J=7.8 and 7.8 Hz); 7.11 (1H, d, J=7.8 Hz); 7.13 (1H, s); 11.60 (1H, br s).

### b) 3-(5-fluoro-4H-quinazolin-3-yl)-pyrrolidine-2,5-dione

Melting point: 184-191° C.

### c) 3-(8-trifluoromethyl-4H-quinazolin-3-yl)-pyrrolidine-2,5-dione

Melting point: 188-190° C.

By replacing the maleimide used in Example 11a with N-methylmaleimide, the following compound was obtained:

### d) 1-methyl-3-(4H-quinazolin-3-yl)-pyrrolidine-2,5-dione

Melting point: 163-164° C.

By replacing the maleimide used in Example 11a with N-methylmaleimide and replacing the 2-aminomethylbenzylamine with 2-aminomethyl-5-fluorobenzylamine, the following compound was obtained:

### e) 7-fluoro-1-methyl-3-(4H-quinazolin-3-yl)-pyrrolidine-2,5-dione

Melting point: 191-193° C.

### Example 12

### a) 3-(5-methoxy-4H-quinazolin-3-yl)-pyrrolidine-2,5-dione

By replacing the 2-aminobenzylamine used in Example 4, stage 6 with 6-methoxy-2-aminobenzylamine and using the procedure described in stages 6 to 8, the target compound was obtained.

Yield: 131 mg (100%), yellowish solid.

Melting point: 222° C. (decomposition)

The 6-methoxy-2-aminobenzylamine required for the preparation of Example 12a was prepared as described in the literature (J. H. Sellstedt et al. J. Med. Chem. 1975, 18, 926-933) starting from 2,6-dinitrobenzonitrile. Reaction with sodium methanolate in anhydrous methanol takes place here in the 1st stage.

2,6-dinitrobenzonitrile can be either acquired commercially or prepared in accordance with the literature (J. R. Beck, J. Org. Chem. 1972, 37, 3224-3226).

By replacing the sodium methanolate in anhydrous methanol used in Example 12a, stage 1 with corresponding sodium alcoholates in the corresponding anhydrous alcohol and using the procedure described above, the following target compounds were obtained:

### b) 3-(5-ethoxy-4H-quinazolin-3-yl)-pyrrolidine-2,5-dione

Melting point: 203-205° C.

### c) 3-(5-pentyloxy-4H-quinazolin-3-yl)-pyrrolidine-2,5-dione

Melting point: 154-160° C.

### d) 3-(5-benzyloxy-4H-quinazolin-3-yl)-pyrrolidine-2,5-dione

Melting point: 100-108° C.

### e) 3-(5-isopropoxy-4H-quinazolin-3-yl)-pyrrolidine-2,5-dione

Melting point: 191-192° C.

### f) 3-(5-(2-methoxyethoxy)-4H-quinazolin-3-yl)-pyrrolidine-2,5-dione

Melting point: 288-290° C.

### Example 13

### a) 3-(5-ethanesulfonyl-4H-quinazolin-3-yl)-pyrrolidine-2,5-dione

### Stage 1: 2-ethylsulfanyl-6-nitrobenzonitrile

A solution of ethanethiol (1.59 g, 1.9 ml, 25.7 mmol) in N,N-dimethylformamide (10 ml) was added to a solution of 2,6-diaminobenzonitrile (4.72 g, 24.4 mmol) and triethylamine (2.60 g, 3.55 ml, 25.7 mmol) in N,N-dimethylformamide (50 ml) at 0° C. The mixture was then stirred at room temperature for 4 h. After addition of water (100 ml), the product which had precipitated out was filtered off, washed with water and dried over phosphorus pentoxide.

Yield: 4.50 g (88%), yellow solid.

Melting point: 126-127° C.

### Stage 2: 2-ethanesulfonyl-6-nitrobenzonitrile

A suspension of periodic acid (8.76 g, 38.4 mmol) in acetonitrile (160 ml) was stirred vigorously at room temperature for 30 min and chromium(VI) oxide (19.2 mg, 0.19 mmol) was then added. The mixture was stirred at room temperature for 5 min, during which it became yellow-orange in color. A solution of the product from stage 1 (2.00 g, 9.6 mmol) in acetonitrile (40 ml) was added dropwise to this mixture, during which a white precipitate precipitated out. The reaction mixture was stirred overnight, the precipitate which had precipitated out was filtered out, and the filtrate was concentrated in vacuo. The residue was taken up in ethyl acetate (150 ml) and the mixture was washed with saturated sodium sulfite solution (three times 50 ml) and saturated sodium chloride solution (60 ml). The organic phase was dried with sodium sulfate and concentrated in vacuo.

Yield: 2.19 g (95%), white solid.

Melting point: 162-163° C.

### Stage 3: 2-amino-6-ethanesulfonylbenzamide

99% strength hydrazine hydrate (500 mg, 486 μl, 10 mmol) was added to a solution of the product from stage 2 (2.10 g. 8.74 mmol) in ethanol (200 ml) at 70° C. Aqueous 50% strength Raney nickel suspension was added in portions to this solution until no further evolution of gas was to be observed. The mixture was then stirred under reflux for 2 hours. Hydrazine hydrate (500 mg, 486 μl, 10 mmol) and Raney nickel suspension were again added and the mixture was heated under reflux for a further 2 hours. After cooling to room temperature, the catalyst was filtered out, the residue on the filter was washed with hot ethanol and ethyl acetate and the filtrate was concentrated in vacuo. The residue obtained was taken up in ethyl acetate (150 ml) and the mixture was dried with sodium sulfate and concentrated again in vacuo.

Yield: 1.88 g (94%), cream-colored solid.

Melting point: 128-134° C.

### Stage 4: 2-aminomethyl-3-ethanesulfonylphenylamine

A 2 M solution or borane-dimethyl sulfide complex in tetrahydrofuran (12.3 ml, 24.6 mmol) was added to a solution of the product from stage 3 (1.88 g, 8.2 mmol) in absolute tetrahydrofuran (100 ml) and the mixture was heated under reflux for 8 hours. Water (3 ml) was then added, the reaction solution was concentrated in vacuo, toluene and methanol (in each case twice) were added to the residue and the mixture was concentrated again each time. The crude product (2.12 g) was purified by flash chromatography with chloroform and 1% triethylamine and later with chloroform/methanol (9:1) and 1% triethylamine.

Yield: 1.41 g (80%), yellowish oil.

1H-NMR (DMSO-d6): 1.10 (3H, t, J=7.3 Hz); 3.30 (2H, q, J=6.8 Hz); 3.98 (2H, s); 5.71 (2H, s, interchangeable); 6.99 (1H, d, J=7.8 Hz); 7.08 (1H, d, J=7.8 Hz); 7.19 (1H, t, J=7.8 Hz). 2 interchangeable signals lie under the HDO signal.

### Stage 5: 3-(2-amino-6-ethanesulfonylbenzylamino)-pyrrolidine-2,5-dione

Maleimide (146 mg, 1.5 mmol) was added to a solution of the product from stage 4 (220 mg, 1 mmol) in tetrahydrofuran (50 ml) and the mixture was stirred at room temperature for 24 hours. The solvent was removed in vacuo and the residue was purified by flash chromatography with chloroform/methanol (9:1).

Yield: 109 mg (35%), white solid.

1H-NMR (DMSO-d6): 1.12 (3H, t, J=6.8 Hz); 2.46-2.49 (1H, m); 2.51-2.58 (1H, m); 2.85 (1H, dd, J=17.6 and 8.8 Hz); 3.22-3.32 (2H, m); 3.79-3.96 (2H, m); 4.17 (1H, dd, J=12.9 and 5.7 Hz); 5.12 (2H, s); 7.00 (1H, d, J=7.8 Hz); 7.11 (1H, d, J=6.8 Hz); 7.22 (1H, t, J=7.8 Hz); 11.20 (1H, s).

### Stage 6: 3-(5-ethanesulfonyl-4H-quinazolin-3-yl)-pyrrolidine-2,5-dione

Orthoformic acid triethyl ester (95 mg, 105 μl, 0.64 mmol) was added to a solution of the product from stage 5 (100 mg, 0.32 mmol) in glacial acetic acid (5 ml) and the mixture was stirred at room temperature for 24 hours. The solvent was removed in vacuo and toluene was repeatedly added to the residue and the mixture concentrated again each time.

Yield: 92 mg (88%), white solid.

Melting point: 210-212° C.

### b) 3-(5-ethanesulfinyl-4H-quinazolin-3-yl)-pyrrolidine-2,5-dione

By using sodium metaperiodate instead of periodic acid and chromium(VI) oxide in Example 13, stage 2 and using the processes described in Example 13, stage 1 and in stage 3 to 6, the title compound is obtained in the form of a mixture of the diastereomers in a ratio of approx. 1:1.

¹H-NMR (DMSO-d₆): 1.02-1.11 (3H, m); 2.50-3.14 (4H, m); 4.17 (0.5H, d, J=14.7 Hz); 4.29 (0.5H, d, J=14.7 Hz); 4.57 (0.5H, d, J=14.7 Hz); 4.74 (0.5H, d, J=13.7 Hz); 4.85-4.95 (1H, m); 6.99-7.08 (1H, m); 7.21 (1H, d, J=6.8 Hz); 7.35-7.44 (2H, m); 11.57, br s)

### c) 3-(5-ethylthio-4H-quinazolin-3-yl)-pyrrolidine-2,5-dione

By omitting stage 2 in Example 13a and using the processes described in Example 13a, stage 1 and in stage 3 to 6, the title compound was obtained.

### Example 14

### 3-(5-nitro-4H-quinazolin-3-yl)-pyrrolidine-2,5-dione

By replacing the 2-aminobenzoic acid used in Example 4, stage 3 with 2-amino-6-nitrobenzoic acid and using the procedure described in stages 3 to 8, the target compound was obtained.

Melting point: 125-129° C.

The preparation of 2-amino-6-nitrobenzoic acid was carried out in accordance with the literature (R. Kahn, Chem. Ber. 1902, 35, 3857-3884; W. S. Saari, J. E. Schwering, J. Heterocycl. Chem. 1986, 23, 1253-1255) via regioselective opening of 3-nitrophthalimide with aqueous potassium hydroxide to give phthalamic acid and subsequent Hofmann degradation with sodium hypobromite to give the 2-amino-6-nitrobenzoic acid.

### Example 15

By replacing the orthoformic acid triethyl ester used in Example 3, stage 5 with orthoacetic acid triethyl ester and using corresponding starting compounds, the following compounds were obtained analogously:

### a) 1-methyl-3-(2-methyl-4H-quinazolin-3-yl)-pyrrolidine-2,5-dione

¹H-NMR (DMSO-d₆): 2.14 (3H, s); 2.90 (3H, s); 2.99 (2H, m); 4.01 (1H, m); 4.48 (1H, d); 5.22 (1H, m); 6.88 (3H, m); 7.11 (1H, m).

### b) 3-(2-methyl-4H-quinazolin-3-yl)-pyrrolidine-2,5-dione

Melting point: 72-75° C.

1H-NMR (DMSO-d6): 2.12 (3H, s); 2.91 (1H, dd, J=17.6 and 8.8 Hz); 2.97 (1H, dd, J=17.6 and 5.9 Hz); 4.02 (1H, d, J=13.7 Hz); 4.52 (1H, d, J=13.7 Hz); 5.22 (1H, dd, J=8.8 and 5.9 Hz); 6.88 (1H, d, J=7.8 Hz); 6.94 (2H, d, J=4.9 Hz); 7.06-7.13 (1H, m); 11.33 (1H, br s).

### c) 3-(5,6-dichloro-2-methyl-4H-quinazolin-3-yl)-pyrrolidine-2,5-dione

Melting point: 161-166° C.

### d) 3-(5,7-dichloro-2-methyl-4H-quinazolin-3-yl)-pyrrolidine-2,5-dione

Melting point: 199-201° C.

### e) 3-(5,7-difluoro-2-methyl-4H-quinazolin-3-yl)-pyrrolidine-2,5-dione

### Free Base:

Melting point: 176-178° C.

The hydrochloride of the title compound was prepared by precipitation from 2-butanone with chlorotrimethylsilane and water in an ice bath:

### 3-(5,7-difluoro-2-methyl-4H-quinazolin-3-yl)-pyrrolidine-2,5-dione hydrochloride

¹H-NMR (DMSO-d₆): 13.6 (1H, s); 11.8 (1H, s); 7.24 (1H, dd, J=9.8 and 9.0 Hz); 7.3 (1H, d, J=9.0 Hz); 5.6 (1H, t, J=8.0 Hz); 4.91 (1H, d, J=15.1 Hz); 4.53 (1H, d, J=15.1 Hz); 3.24 (1H, dd, J=18.1 and 6.8 Hz); 3.01 (1H, dd, J=18.1 and 9.0 Hz); 2.57 (3H, s).

### f) 3-(5,8-dichloro-2-methyl-4H-quinazolin-3-yl)-pyrrolidine-2,5-dione

Melting point: 186-191° C.

### g) 3-(5-benzyloxy-2-methyl-4H-quinazolin-3-yl)-pyrrolidine-2,5-dione

¹H-NMR (DMSO-d₆): 1.90 (4H, s, HOAc); 2.10 (3H, s); 2.92 (1H, dd, J=17.6 and 8.8 Hz); 3.05 (1H, dd, J=18.6 and 5.9 Hz); 4.09 (1H, d, J=13.7 Hz); 4.43 (1H, d, J=14.7 Hz); 5.11 (2H, s); 5.25 (1H, dd, J=8.8 and 5.9 Hz); 6.49 (1H, d, J=7.8 Hz); 6.67 (1H, d, J=7.8 Hz); 7.02 (1H, t, J=7.8 Hz); 7.09-7.44 (5H, m); ca. 11.7 (1H, very br s).

### h) 3-(5-bromo-2-methyl-4H-quinazolin-3-yl)-pyrrolidine-2,5-dione

¹H-NMR (DMSO-d₆): 2.11 (3H, s); 2.94 (1H, dd, J=18.0 and 9.3 Hz); 3.10 (1H, dd, J=17.6 and 5.9 Hz); 4.07 (1H, d, J=13.7 Hz); 4.48 (1H, d, J=13.7 Hz); 5.27 (1H, dd, J=8.8 and 5.9 Hz); 6.86 (1H, t, J=8.8 Hz); 7.07 (1H, t, J=8.3 Hz); 7.20 (1H, d, J=7.8 Hz); 11.92 (1H, s).

### i) 3-(5-chloro-2-methyl-4H-quinazolin-3-yl)-pyrrolidine-2,5-dione

Melting point: 163-170° C.

### j) 3-(5-chloro-7-fluoro-2-methyl-4H-quinazolin-3-yl)-pyrrolidine-2,5-dione

### Free Base:

Melting point: 179-181° C.

### 3-(5-chloro-7-fluoro-2-methyl-4H-quinazolin-3-yl)-pyrrolidine-2,5-dione hydrochloride

The hydrochloride of the title compound was obtained in the conventional manner.

### k) 3-(5-ethoxy-2-methyl-4H-quinazolin-3-yl)-pyrrolidine-2,5-dione

Melting point: 130-133° C.

### l) 3-(5-fluoro-2-methyl-4H-quinazolin-3-yl)-pyrrolidine-2,5-dione

Melting point: 185° C.

### m) 3-(5-isopropoxy-2-methyl-4H-quinazolin-3-yl)-pyrrolidine-2,5-dione

Melting point: 116-119° C.

### n) 3-(2,5-dimethyl-4H-quinazolin-3-yl)-pyrrolidine-2,5-dione

Melting point: 200-201° C.

### o) 3-(5-methoxy-2-methyl-4H-quinazolin-3-yl)-pyrrolidine-2,5-dione

Melting point: 122° C.

### p) 3-(2-methyl-5-nitro-4H-quinazolin-3-yl)-pyrrolidine-2,5-dione

Melting point: >260° C. decomposition

### q) 3-(2-methyl-5-pentyloxy-4H-quinazolin-3-yl)-pyrrolidine-2,5-dione

Melting point: 154-156° C.

### r) 3-(5-ethylthio-2-Methyl-4H-quinazolin-3-yl)-pyrrolidine-2,5-dione

### s) 3-(5-ethanesulfonyl-2-methyl-4H-quinazolin-3-yl)-pyrrolidine-2,5-dione

1H-NMR (DMSO-d6): 1.08 (3H, t, J=7.3 Hz); 2.15 (3H, s); 3.00 (2H, d, J=6.8 Hz); 3.16-3.30 (2H, m); 4.48 (1H, d, J=14.7 Hz); 4.67 (1H, d, J=14.7 Hz); 5.26 (1H, t, J=7.3 Hz); 7.23 (1H, d, J=7.8 Hz); 7.43 (1H, t, J=7.8 Hz); 7.49 (1H, d, J=7.8 Hz).

### t) 3-(2-methyl-4H-benzo[g]quinazolin-3-yl)-pyrrolidine-2,5-dione

Melting point: 106-110° C.

### u) 3-(6,7-difluoro-2-methyl-4H-quinazolin-3-yl)-pyrrolidine-2,5-dione

Melting point: 183-185° C.

### v) 3-(6,7-dimethoxy-2-methyl-4H-quinazolin-3-yl)-pyrrolidine-2,5-dione

Melting point: 200-202° C.

### w) 3-(6,8-dichloro-2-methyl-4H-quinazolin-3-yl)-pyrrolidine-2,5-dione

Melting point: 180-182° C.

### x) 3-(6-benzyloxy-2-methyl-4H-quinazolin-3-yl)-pyrrolidine-2,5-dione

Melting point: 132-143° C.

### y) 3-(6-chloro-2-methyl-4H-quinazolin-3-yl)-pyrrolidine-2,5-dione

Melting point: 195-203° C.

### z) 3-(2-methyl-6-nitro-4H-quinazolin-3-yl)-pyrrolidine-2,5-dione

Melting point: 170-175° C.

### aa) 3-(2-methyl-7-trifluoromethyl-4H-quinazolin-3-yl)-pyrrolidine-2,5-dione

Melting point: 205-206° C.

### bb) 3-(7-chloro-2-methyl-4H-quinazolin-3-yl)-pyrrolidine-2,5-dione

Melting point: 153-155° C.

### cc) 3-(7-fluoro-2-methyl-4H-quinazolin-3-yl)-pyrrolidine-2,5-dione

¹H-NMR (DMSO-d₆) 2.12 (3H, s); 2.90 (1H, dd, J=17.6 and 8.8 Hz); 3.00 (1H, dd, J=18.0 and 6.0 Hz); 4.02 (1H, d, J=13.7 Hz); 4.51 (1H, d, J=13.7 Hz); 5.25 (1H, dd, J=8.8 and 6.8 Hz); 6.65 (1H, d, J=10.8 Hz); 6.77 (1H, dt, J=8.8 and 2.0 Hz); 6.98 (1H, t, J=6.8 Hz); approx. 11.8 (1H, very br s).

### dd) 3-(7-methoxy-2-methyl-4H-quinazolin-3-yl)-pyrrolidine-2,5-dione

¹H-NMR (DMSO-d₆): 2.12 (3H, s); 2.90 (1H, dd, J=18.0 and 9.0 Hz); 2.98 (1H, dd, J=18.0 and 6.0 Hz); 3.69 (3H, s); 3.98 (1H, d, J=13.7 Hz); 4.45 (1H, d, J=13.7 Hz); 5.23 (1H, dd, J=8.8 and 6.8 Hz); 6.46 (1H, d, J=2.0 Hz); 6.55 (1H, dd, J=7.8 and 2.0 Hz); 6.86 (1H, d, J=7.8 Hz); 11-12 (1H, very br s).

### ee) 3-(2-methyl-7-nitro-4H-quinazolin-3-yl)-pyrrolidine-2,5-dione

Melting point: 203-210° C.

### ff) 3-(8-bromo-2,6-dimethyl-4H-quinazolin-3-yl)-pyrrolidine-2,5-dione

Melting point: 145-150° C.

### gg) 3-(2-methyl-8-trifluoromethyl-4H-quinazolin-3-yl)-pyrrolidine-2,5-dione

¹H-NMR (DMSO-d₆): 2.16 (3H, s); 2.91 (1H, dd, J=18.6 and 8.8 Hz); 3.00 (1H, dd, J=18.6 and 6.8 Hz); 4.10 (1H, d, J=13.7 Hz); 4.59 (1H, d, J 13.7 Hz); 5.26 (1H, dd, J=8.6 and 6.8 Hz); 7.07 (1H, t, J=7.8 Hz); 7.21 (1H, d, J=7.8 Hz); 7.42 (1H, d, J=7.8 Hz); 11.60 (1H, br s).

### hh) 3-(8-chloro-2-methyl-4H-quinazolin-3-yl)-pyrrolidine-2,5-dione

Melting point: 176-178° C.

### ii) 3-(8-methoxy-2-methyl-4H-quinazolin-3-yl)-pyrrolidine-2,5-dione

¹H-NMR (DMSO-d₆): 1.90 (6H, s, HOAc); 2.13 (3H, s); 2.90 (1H, dd, J=18.6 and 8.8 Hz); 2.96 (1H, dd, J=18.6 and 6.8 Hz); 3.72 (3H, s); 3.98 (1H, d, J=13.7 Hz); 4.46 (1H, d, J=13.7 Hz); 5.22 (1H, dd, J=8.8 and 6.8 Hz); 6.53 (1H, d, J=7.8 Hz); 6.77 (1H, d, J=8.8 Hz); 6.92 (1H, t, J=7.8 Hz).

### jj) 3-(5-(2-methoxyethoxy)-2-methyl-4H-quinazolin-3-yl) pyrrolidine-2,5-dione

1H-NMR (DMSO-d6): 2.10 (3H, s); 2.94-3.00 (2H, m); 3.28 (3H, s); 3.59-3.62 (2H, m); 4.01 (1H, d, J=13.7 Hz); 4.05-4.08 (2H, m); 4.37 (1H, d, J=13.7 Hz); 5.24 (1H, dd, J=8.8 and 6.8 Hz); 6.50 (1H, d, J=7.8 Hz); 6.64 (1H, d, J=7.8 Hz); 7.05 (1H, t, J=7.8 Hz); 11.52 (1 Hbrs).

### Example 16

### a) 3-(7-amino-4H-quinazolin-3-yl)-pyrrolidine-2,5-dione hydrochloride

### Stage 1: 3-(7-amino-4H-quinazolin-3-yl)-pyrrolidine-2,5-dione

A solution of the nitro compound from Example 14 (93 mg, 0.34 mmol) in acetic acid (25 ml) was hydrogenated with palladium on active charcoal (20 mg, 10%) under a hydrogen atmosphere of 3 bar at room temperature for 20 min. The reaction mixture was then filtered and concentrated in vacuo.

Yield: 80 mg (99%), brownish oil.

¹H-NMR (DMSO-d₆): 2.90 (1H, dd, J=16.6 and 9.7 Hz); 3.48 (1H, t, J=5.3 Hz); 3.96 (1H, d, J=12.7 Hz); 4.42 (1H, d, J=12.7 Hz); 4.82 (1H, m); 6.22 (1H, m); 6.58 (1H, m); 7.06-7.30 (2H, m).

### Stage 2: 3-(7-amino-4H-quinazolin-3-yl)-pyrrolidine-2,5-dione hydrochloride

A solution of hydrogen chloride in diethyl ether (1 ml) was added to a solution of the amino compound from stage 1 (93 mg, 0.34 mmol) in methanol (3 ml), while stirring, a pale precipitate precipitating out immediately. After addition of diethyl ether, the mixture was decanted, diethyl ether was added again and the mixture was decanted. The residue was dried in vacuo.

Yield: 50 mg (53%), yellow solid.

¹³C-NMR (DMSO-d₆): 32.3; 43.2; 62.1; 100.9; 106.9; 111.8; 118.1; 127.9; 130.0; 151.8; 173.6; 174.8.

By replacing the starting material used in Example 15a, stage 1 with corresponding starting compounds and using the procedure described in stages 1 to 2, the following compounds were obtained analogously:

### b) 3-(7-amino-2-methyl-4H-quinazolin-3-yl)-pyrrolidine-2,5-dione hydrochloride

¹H-NMR (DMSO-d₆): 2.57 (3H, s); 2.98-3.10 (2H, m); 4.47 (1H, d, J=14.7 Hz); 4.90 (1H, d, J=14.7 Hz); 5.53 (1H, t, J=7.8 Hz); 7.10 (1H, d, J=7.8 Hz); 7.19 (2H, d, J=6.8 Hz); 11.92 (1H, s); 13.34 (1H, s).

### c) 3-(6-amino-4H-quinazolin-3-yl)-pyrrolidine-2,5-dione hydrochloride

¹H-NMR (DMSO-d₆): 2.98 (1H, dd, J=18.0 and 9.0 Hz); 3.18 (1H, dd, J=17.6 and 5.9 Hz); 4.56 (1H, d, J=15.6 Hz); 4.99 (1H, d, J=8.8 Hz); 5.25 (1H, dd, J=9.8 and 5.8 Hz); 7.02 (1H, s); 7.19 (1H, d, J=8.8 Hz); 7.24 (1H, d, J=8.8 Hz); 8.69 (1H, s); 11.86 (1H, s); 13.10 (1H, very br s).

### d) 3-(6-amino-2-methyl-4H-quinazolin-3-yl)-pyrrolidine-2,5-dione hydrochloride

¹H-NMR (DMSO-d₆): 2.56 (3H, s); 2.99 (1H, dd, J=18.0 and 9.0 Hz); 3.18 (1H, dd, J=18.6 and 5.9 Hz); 4.51 (1H, d, J=15.6 Hz); 4.95 (1H, d, J=15.6 Hz); 5.53 (1H, dd, J=8.9 and 6.8 Hz); 7.07 (1H, s); 7.22 (1H, d, J=7.8 Hz); 7.32 (1H, d, J=7.8 Hz); 11.90 (1H, s); 13.3 (1H, s).

### e) 3-(5-amino-4H-quinazolin-3-yl)-pyrrolidine-2,5-dione hydrochloride

¹H-NMR (DMSO-d6): 2.96 (1H, dd, J=17.6 and 9.0 Hz), 3.20 (1H, dd, J=17.6 and 5.9 Hz); 4.27 (1H, d, J=14.7 Hz); 4.87 (1H, d, J=15.6 Hz); 5.27 (1H, dd, J=9.0 and 6.0 Hz); 6.61 (1H, d, J=7.8 Hz); 6.78 (1H, d, J=7.8 Hz); 6.84 (2H, very br s); 7.13 (1H, t, J=7.8 Hz); 11.92 (1H, s); 13.03 (1H, s).

### f) 3-(5-amino-2-methyl-4H-quinazolin-3-yl)-pyrrolidine-2,5-dione hydrochloride

¹H-NMR (DMSO-d₆): 2.33 (3H, s); 2.99 (1H, dd, J=18.0 and 9.0 Hz); 3.26 (1H, dd, J=18.0 and 6.0 Hz), 4.30 (1H, d, J=14.7 Hz); 4.83 (1H, d, J=14.7 Hz); 5.55 (1H, dd, J=9.0 and 6.8 Hz); 6.49-7.04 (2H, very br s); 6.73 (1H, d, J=7.8 Hz); 6.79 (1H, d, J=7.8 Hz); 7.14 (1H, t, J=7.8 Hz); 11.90 (1H, s); 13.02 (1H, s).

### Example 17

### a) 3-(5-hydroxy-4H-quinazolin-3-yl)-pyrrolidine-2,5-dione hydrochloride

### Stage 1: 3-(5-hydroxy-4H-quinazolin-3-yl)-pyrrolidine-2,5-dione

10% palladium on active charcoal (144 mg) was added to a solution of the product from Example 12e (550 mg, 1.64 mmol) in glacial acetic acid (50 ml) under argon and hydrogenation was then carried out at room temperature under 3 bar of hydrogen for 3 h. The reaction solution was filtered and concentrated in vacuo.

Yield: 606 mg, brown oil.

¹H-NMR (DMSO-d₆): 2.83 (1H, dd, J=18.6 and 8.8 Hz); 3.02 (1H, dd, J=17.6 and 5.9 Hz); 3.98 (1H, d, J=13.7 Hz); 4.45 (1H, d, J=14.7 Hz); 4.85 (1H, dd, J=9.8 and 5.9 Hz); 6.35 (1H, d, J=7.8 Hz); 6.48 (1H, d, J=7.8 Hz); 6.91 (1H, t, J=7.8 Hz); 7.09 (1H, s).

### Stage 2: 3-(5-hydroxy-4H-quinazolin-3-yl)-pyrrolidine-2,5-dione hydrochloride

A solution of hydrogen chloride in diethyl ether (5 ml) was added to a solution of the product from stage 1 (650 mg, 2.01 mmol) in glacial acetic acid (1 ml). The supernatant was decanted and diethyl ether was repeatedly added to the solid and decanted again each time. The solid product was dried over potassium hydroxide in a desiccator.

Yield: 373 mg (61%), white solid.

Melting point: 182-188° C.

By replacing the product from Example 12e in Example 17a by the products from Example 9b, 15f and 15v and using the procedure described in Example 17a, stages 1 to 2, the following compounds were obtained analogously:

### b) 3-(6-hydroxy-2-methyl-4H-quinazolin-3-yl)-pyrrolidine-2,5-dione hydrochloride

¹H-NMR (DMSO-d₆): approx. 2.5 (3H, s); 3.01 (1H, dd, J=18.0 and 9.4 Hz); 3.16 (1H, dd, J=18.4 and 6.7 Hz); 4.39 (1H, d, J=14.9 Hz); 4.84 (1H, d, J=15.6 Hz); 5.47 (1H, dd, J=9.4 and 6.3 Hz); 6.60 (1H, d, J=2.3 Hz); 6.75 (1H, dd, J=8.6 and 3.1 Hz); 7.05 (1H, dd, J=5.8 and 2.8 Hz); 9.92 (1H, s); 11.84 (1H, s); approx. 12.6 (1H very br s).

### c) 3-(6-hydroxy-2-methyl-4H-quinazolin-3-yl)-pyrrolidine-2,5-dione hydrochloride

¹H-NMR (DMSO-d₆): approx. 2.5 (3H, s, overlapped by DMSO signal); 3.01 (1H, dd, J=18.0 and 9.4 Hz); 3.16 (1H, dd, J=18.4 and 6.7 Hz); 4.39 (1H, d, J=14.9 Hz); 4.84 (1H, d, J=15.6 Hz); 5.47 (1H, dd, J=9.4 and 6.3 Hz); 6.60 (1H, d, J=2.3 Hz); 6.75 (1H, dd, J=8.6 and 3.1 Hz); 7.05 (1H, dd, J=5.8 and 2.8 Hz); 9.92 (1H, s); 11.84 (1H, s); approx. 12.6 (1H very br s).

### d) 3-(5-hydroxy-2-methyl-4H-quinazolin-3-yl)-pyrrolidine-2,5-dione hydrochloride

1H-NMR (DMSO-d6): 1.90 (3H, s, HOAc); 2.53 (3H, s); 3.02 (1H, dd, J=18.6 and 8.8 Hz); 3.25 (1H, dd, J=18.6 and 5.9 Hz); 4.31 (1H, d, J=14.7 Hz); 4.70 (1H, d, J=15.7); 5.51 (1H, dd, J=8.8 and 6.8 Hz); 6.69 (1H, d, J=7.8 Hz); 6.77 (1H, d, J=7.8 Hz); 7.15 (1H, t, J=7.8 Hz); 10.48 (1H, s); 11.91 (1H, s); 12.00 (1H, br s); 12.93 (1H, s).

### Example 18

The position of the thiocarbonyl group has not yet been proved. It follows as a conclusion of analogy from the investigations of the thionation of 2-benzyloxycarbonyl-aminoglutarimide, in which the carbonyl group with the least steric hindrance reacted the fastest.

### a) 3-(7-fluoro-4H-quinazolin-3-yl)-5-thioxopyrrolidin-2-one hydrobromide

### Stage 1: (2-{[benzyloxycarbonyl-(2,5-dioxopyrrolidin-3-yl)-amino]-methyl}-5-fluorophenyl)-carbamic acid benzyl ester

A solution of the product from Example 3, stage 4 (9.83 g, 41 mmol), carbonic acid benzyl ester 2,5-dioxopyrrolidin-1-yl ester (22.0 g, 88 mmol) and 4-N,N-dimethylaminopyridine (100 mg) in anhydrous tetrahydrofuran (50 ml) was stirred at room temperature for 24 h. The reaction mixture was concentrated in vacuo, the residue was dissolved in ethyl acetate and the organic phase was washed twice with water. The organic phase was dried with sodium sulfate and concentrated in vacuo. Half of the residue was purified by flash chromatography with ethyl acetate/cyclohexane (2:3), whereby 5.54 g of a mixture of mono- and di-benzyloxycarbonyl-substituted product were obtained in the ratio of approx. 1:1. Repeated reaction with carbonic acid benzyl ester 2,5-dioxopyrrolidin-1-yl ester and subsequent repeated flash chromatography of the reaction mixtures and mixed fractions with chloroform/isopropanol, (99:1) gave the title compound.

Yield: 6.16 g (29%), colorless foam.

¹H-NMR (DMSO-d₆): 2.61 (1H, dd, J=17.6 and 5.9 Hz); 2.74 (1H, dd, J=17.6 and 9.8 Hz); 4.20-4.62 (3H, m); 5.00-5.20 (4H, m); 6.99 (1H, dt, J=8.8 and 2.9 Hz); 7.18-7.52 (12H, m); 9.27 and 9.30 (1H, 2 s); 11.25 and 11.28 (1H, 2 s).

### Stage 2: (2-{[benzyloxycarbonyl-(2-oxo-5-thioxopyrrolidin-3-yl)amino]methyl}-5-fluorophenyl)-carbamoyl acid benzyl ester

A mixture of the product from stage 1 (2.1 g, 4.5 mmol) and tetraphosphorus decasulfide (4.0 g, 18 mmol, based on P₂S₂) in tetrahydrofuran (60 ml) was treated in a microwave oven under an argon atmosphere at 90° C. for 40 min. The precipitate which had precipitated out was filtered off and washed with hot chloroform. Silica gel 60 (0.2-0.5 mm) was added to the combined filtrates, the suspension was concentrated in vacuo and the residue was purified by flash chromatography with ethyl acetate/cyclohexane (1:2).

Yield: 1.44 g (62%), yellow solid.

¹H-NMR (DMSO-d₆): 2.88-3.24 (2H, m); 4.20-4.68 (3H, m); 4.92-5.70 (4H, m); 6.92-7.52 (13H, m); 9.27 (1H, s); 12.78 and 12.84 (1H, 2 s).

The ¹³C-NMR spectrum (DMSO-d₆) shows characteristic signals at 211.3 ppm (C═S) and at 177.7 ppm (C═O).

### Stage 3: 3-(2-amino-4-fluorobenzylamino)-5-thioxopyrrolidin-2-one dihydrobromide

A 33% solution of hydrogen bromide in acetic acid (3 ml) was added to a suspension of the product from stage 2 (770 mg, 1.5 mmol) in acetic acid (2 ml) and methylene chloride (3 ml). The reaction mixture was stirred at room temperature for 3 h, diethyl ether (approx. 20 ml) was then added and the mixture was left to stand overnight. The solid which had precipitated out was filtered out, washed with diethyl ether and dried in vacuo.

Yield: 570 mg (93%), beige-colored solid.

Melting point: 150-153° C.

### Stage 4: 3-(7-fluoro-4H-quinazolin-3-yl)-5-thioxopyrrolidin-2-one hydrobromide

A mixture of the product from stage 3 (200 mg, 0.48 mmol) in acetic acid (5 ml), methylene chloride (5 ml) and orthoformic acid triethyl ester (142 mg 157 μl, 0.96 mmol) was stirred at room temperature. After 3 h, further orthoformic acid triethyl ester (71 mg, 80 μl, 0.48 mmol) was added to the reaction mixture, the mixture was stirred at room temperature for 2 h and concentrated in vacuo and the residue was dried in vacuo.

Yield: 175 mg (90%), yellowish solid.

Melting point: 202-208° C. (decomposition)

### b) 3-(4H-quinazolin-3-yl)-5-thioxopyrrolidin-2-one hydrobromide

### Stage 1: (2-{[benzyloxycarbonyl-(2,5-dioxopyrrolidin-3-yl)amino]methyl}-phenyl)carbamoyl acid benzyl ester

A solution of carbonic acid benzyl ester 2,5-dioxopyrrolidin-1-yl ester (10.2 g, 41 mmol) in tetrahydrofuran (10 ml) was added to a solution of 3-(2-amino-benzylamino)-pyrrolidine-2,5-dione (4.38 g, 20 mmol) and 4-N,N-dimethylaminopyridine (spatula-tip) in tetrahydrofuran (40 ml). The solution was subsequently stirred at room temperature overnight and then concentrated in vacuo. The residue was dissolved in chloroform and the solution was washed with water (3×100 ml). The organic phase was dried with sodium sulfate and concentrated in vacuo. The residue (10.7 g) was purified by flash chromatography with ethyl acetate/cyclohexane (1:1).

Yield: 6.45 g (66%), white solid.

Melting point: 61-64° C.

### Stage 2 to 4

By replacing (2-{[benzyloxycarbonyl-(2,5-dioxopyrrolidin-3-yl)-amino]-methyl}-5-fluorophenyl)-carbamic acid benzyl ester in Example 16a, stage 2 by (2-{[benzyloxycarbonyl-(2,5-dioxopyrrolidin-3-yl)amino]methyl}phenyl)-carbamoyl acid benzyl ester and using the processes used in Example 16a, stage 2 to 4, the target compound was obtained:

Melting point: 215-217° C.

### Example 19

### a) 3-methyl-3-(4H-quinazolin-3-yl)-pyrrolidine-2,5-dione

### Stage 1: 3-methylpyrrole-2,5-dione

A solution of hexamethyldisilazane (80.7 g, 104.8 ml, 500 mmol) in methanol was added dropwise to a solution of citraconic anhydride (5.60 g, 4.49 ml, 50 mmol) in N,N-dimethylformamide (170 ml) at 0° C. The reaction solution was stirred at room temperature for 20 h, thereafter methanol (100 ml) was added and the mixture was stirred for a further 20 min. The solvent was concentrated in vacuo and the residue was taken up in a mixture of ethyl acetate/water (150 ml:50 ml). The organic phase was separated off, washed with water (2×50 ml), sodium bicarbonate solution (50 ml) and with water again (50 ml) and then dried with sodium sulfate. The solvent was concentrated and the residue was dried in vacuo.

Yield: 2.30 g (40%), white solid

Melting point: 100-102° C. [lit. 101-104° C. (K. R. Shah, C. DeWitt Blanton, J. Org. Chem. 1982, 47, 502)].

### Stage 2: 3-(2-aminobenzylamino)-3-methylpyrrolidine-2,5-dione

A solution of the product from stage 1 (1.94 g, 17.5 mmol) and 2-aminobenzylamine (1.49 g, 12.2 mmol) in ethyl acetate (44 ml) was stirred at 50° C. for 48 h and thereafter concentrated in vacuo. The residue (3.8 g) was fractionated by flash chromatography with chloroform/methanol (9:1).

Yield: 1.07 g (37%), white solid.

Melting point: 129-134° C.

### Stage 3: 3-methyl-3-(4H-quinazolin-3-yl)-pyrrolidine-2,5-dione

Orthoformic acid triethyl ester (296 mg, 328 μl, 2 mmol) was added to a solution of the product from stage 2 (233 mg, 1.0 mmol) in acetic acid (10 ml) and the mixture was stirred at room temperature for 5 h. The reaction mixture was concentrated and dried in vacuo. Diethyl ether was added to the residue, the mixture was left to stand at 4° C. for 20 h and the solid which had precipitated out was filtered out, washed with diethyl ether and dried in vacuo.

Yield: 206 mg (68%), white solid.

Melting point: 112-125° C.

### b) 3-(5-bromo-4H-quinazolin-3-yl)-3-methyl-pyrrolidine-2,5-dione

By replacing the 2-aminobenzylamine in Example 19a, stage 1 by 2-aminomethyl-3-bromo-phenylamine and using the processes described in Example 19a, stage 2 and 3, the target compound was obtained:

Melting point: 124-125° C.

### Example 20

### a) 3-(2-oxo-1,4-dihydro-2H-quinazol in-3-yl)-pyrrolidine-2,5-dione

### Stage 1: 3-(2-amino-benzylamino)-pyrrolidine-2,5-dione

A mixture of maleimide (485 mg, 5 mmol) and 2-aminobenzylamine (671 mg, 5.5 mmol) in ethyl acetate (7 ml) was stirred at room temperature for 20 h. The solution was concentrated in vacuo and the residue was purified by flash chromatography with chloroform/methanol (97:3).

Yield: 1.09 g (100%), viscous oil

¹H-NMR (DMSO-d₆): 2.40 (1H, dd, J=17.6 and 4.9); 2.65 (1H, br s); 2.75 (1H, dd, J=17.6 and 7.8 Hz); 3.60-3.80 (3H, m); 5.15 (2H, s); 6.49 (1H, dd, J=7.8 and 7.8 Hz); 6.59 (1H, d, J=7.8 Hz); 6.93-6.98 (2H, m); 11.10 (1H, s).

### Stage 2: 3-(2-oxo-1,4-dihydro-2H-quinazolin-3-yl)-pyrrolidine-2,5-dione

A solution of the product from stage 1 (300 mg, 1.37 mmol) and N,N′-carbonyldiimidazole (447 mg, 2.74 mmol) in tetrahydrofuran (40 ml) was boiled under reflux for 3 h, stirred at room temperature overnight and boiled under reflux again for 5 h, carbonyldiimidazole was again added (447 mg, 2.74 mmol) and the mixture was boiled under reflux for a further 3 hours. The reaction mixture was concentrated in vacuo and the residue was purified by flash chromatography with chloroform/isopropanol (95:5).

Yield: 264 mg (79%), white solid.

Melting point: 251° C.

### b) 3-(7-fluoro-2-oxo-1,4-dihydro-2H-quinazolin-3-yl)-pyrrolidine-2,5-dione

By replacing the 2-aminobenzylamine by 2-(aminomethyl)-5-fluorobenzylamine in Example 20a, stage 1 and using the processes described in Example 20a, stage 1 to 2, the title compound was obtained:

Melting point: 283-285° C.

### c) 3-(5,7-difluoro-2-oxo-1,4-dihydro-2H-quinazolin-3-yl)-pyrrolidine-2,5-dione

By replacing the 2-aminobenzylamine by 2-(aminomethyl)-3,5-difluorobenzylamine in Example 20a, stage 1 and using the processes described in Example 20a, stage 1 to 2, the title compound was obtained:

### d) 3-(2-oxo-1,4-dihydro-2H-quinazolin-3-yl)-4-phenylpyrrolidine-2,5-dione

### Stage 1: 3-phenylpyrrole-2,5-dione

A solution of aniline (4.66 g, 50 mmol) in concentrated hydrochloric acid (15 ml) and water (10 ml) was cooled to 0° C., with vigorous stirring, and ice (10 g) was added. A solution of sodium nitrite (3.45 g, 50 mmol) in water (15 ml) was added dropwise to the emulsion formed such that the internal temperature did not rise above 5° C. The cold suspension was added in one portion to a suspension, cooled to 0° C., of maleimide (5.82 g, 60 mmol) in acetone (15 ml). The pH of this mixture was adjusted to 3 by addition of solid sodium acetate. Copper(II) chloride (1.0 g) was then added, followed by acetone in an amount sufficient for a clear solution to form. After stirring at 0° C. for 30 min, the mixture was heated to 40° C., during which evolution of nitrogen started. The mixture was stirred at this temperature overnight, before acetone was stripped off in vacuo at a maximum bath temperature of 20° C. The residue was extracted with ethyl acetate (three times 50 ml). The combined organic extracts were dried with sodium sulfate and concentrated in vacuo and the residue was purified by flash chromatography with cyclohexane/ethyl acetate (4:1).

Yield: 1.48 g (17%), yellow solid.

Melting point: 160-164° C.

### Stage 2: 3-(2-aminobenzylamino)-4-phenylpyrrolidine-2,5-dione

2-aminobenzylamine (843 mg, 6.9 mmol) was added to a solution of the product from stage 1 (1.20 g, 6.9 mmol) in ethyl acetate (20 ml) and the mixture was stirred at room temperature for 24 h. The solid which had precipitated out was filtered off and dried in vacuo.

Yield: 403 mg (32%), white solid.

Melting point: 167-169° C.

### Stage 3: 3-(2-oxo-1,4-dihydro-2H-quinazolin-3-yl)-4-phenylpyrrolidine-2,5-dione

The product from stage 2 (1.10 g, 3.72 mmol) was added in portions to a solution, boiling under reflux, of N,N′-carbonyldiimidazole (1.21 g, 7.44 mmol) in tetrahydrofuran (100 ml). The reaction mixture was then heated under reflux for 6 h and, after cooling to room temperature, was concentrated in vacuo. The residue was purified by means of flash chromatography with chloroform/methanol (9:1) and subsequent flash chromatography with ethyl acetate/cyclohexane (2:1).

Yield: 230 mg (19%), yellow solid, which deliquesces in air.

Melting point: 226-236° C.

### e) 3-(2-thio-1,4-dihydro-2H-quinazolin-3-yl)-pyrrolidine-2,5-dione

By replacing the carbonyldiimidazole in Example 20a, stage 2 by N,N′-thiocarbonyldiimidazole and using the processes described in Example 20a, stage 1 to 2, the title compound was obtained:

Melting point: 235-239° C.

### Example 21

### 3-(2-(methylthio)-4H-quinazolin-3-yl)pyrrolidine-2,5-dione hydroiodide

Methyl iodide (527 mg, 0.231 ml, 3.7 mmol) was added to a solution of the product from Example 20d (194 mg, 0.74 mmol) in absolute tetrahydrofuran (20 ml) and the mixture was stirred at room temperature for 7 d. The reaction solution was then concentrated in vacuo.

Yield: 300 mg (100%), brown solid

¹H-NMR (DMSO-d₆): 2.60 (3H, s); 2.80-3.05 (2H, m); 4.16-4.30 (1H, m); 4.58-4.75 (1H, m); 5.50 (1H, br s); 6.95-7.18 (3H, m); 7.20-7.36 (1H, m); 11.69 (1H, s).

### Example 22

### 3-(2-(dimethylamino)-4-quinazolin-3-yl)pyrrolidine-2,5-dione hydroiodide

2 M dimethylamine solution in tetrahydrofuran (0.17 ml, 0.34 mmol) was added to a solution of the product from Example 21 (138 mg, 0.34 mmol) in absolute tetrahydrofuran (10 ml) and the mixture was stirred at room temperature for 2 days. The reaction solution was then concentrated in vacuo.

Yield: 121 mg (89%), brown solid.

¹H-NMR (DMSO-d₆): 2.45 (3H, s); 2.54 (3H, s); 2.90 (2H, dd, J=11.7 and 5.9 Hz); 4.05 (1H, d, J=13.7 Hz); 4.53 (1H, dd, J=13.7 Hz); 5.34 (1H, dd, J=8.8 and 5.9 Hz); 6.80-7.05 (3H, m); 7.12-7.20 (1H, m); 8.01-8.30 (1H, br s); 11.58 (1H, s).

### Example 23

### 3-(4H-Quinazolin-3-yl)-pyrrolidin-2-one

### Stage 1: 3-aminopyrrolidin-2-one

The synthesis was carried out in accordance with the literature (R. Pellegata et al., Synthesis, 1978, 614-616).

### Stage 2: 3-(4H-quinazolin-3-yl)-pyrrolidin-2-one

2-formylamino-benzaldehyde (1.49 g, 10 mmol, for the preparation process see WO 03/053956 A1) was added to a solution of the product from stage 1 (1.00 g, 10 mmol) in absolute 1,2-dichloroethane (150 ml) and the mixture was stirred at room temperature for 15 min. Acetic acid (601 mg, 572 μl, 10 mmol) and sodium triacetoxyborohydride (3.04 g, 14.3 mmol) were added to the mixture and the mixture was stirred at room temperature overnight. The reaction mixture was adjusted to pH ˜9 with 2 N sodium hydroxide solution. After separation of the phases, the aqueous phase was extracted with methylene chloride (two times 20 ml). The combined organic phases were dried with sodium sulfate and concentrated in vacuo. The residue was purified by flash chromatography with chloroform/methanol (9:1) and methanol. The fraction (123 mg) eluted by methanol was purified again by means of flash chromatography with chloroform/methanol (3:7).

Yield: 78 mg (3.6%), yellowish solid.

Melting point: 222-225° C.

### Example 24

### a) 3-(4H-quinazolin-3-yl)-pyrrolidin-2-one

A solution of 3,4-dihydroquinazoline (505 mg, 2 mmol), maleimide (292 mg, 3 mmol) and triethylamine (607 mg, 832 μl, 6 mmol) in N,N-dimethylformamide (10 ml) was stirred at room temperature overnight. The mixture was then concentrated in vacuo, the residue was taken up in toluene, the mixture was concentrated again and the residue was purified by flash chromatography with ethyl acetate/methanol (2:1).

Yield: 341 mg (74%), beige-colored foam. The ¹H-NMR spectrum was identical to that of Example 11a.

### b) 3-(2-methyl-4H-quinazolin-3-yl)-pyrrolidine-2,5-dione

By replacing the 3,4-dihydroquinazoline by 2-methyl-3,4-dihydroquinazoline in Example 24a and using the processes described there, the title compound was obtained. The ¹H-NMR spectrum was identical to that of Example 15b.

### Investigation of the Immunomodulatory Activity

Stimulation of human monocytes with lipopolysaccharide for secretion of IL-10 and IL-12:

Human monocytes were isolated from peripheral blood mononuclear cells (PBMC), which were obtained from heparinized whole blood by means of a Ficoll density gradient centrifugation. For this, the PBMC were incubated with a monoclonal antibody which is directed against the monocyte-specific surface molecule CD14 and to which superparamagnetic microbeads (Miltenyi Biotech, Bergisch Gladbach) are coupled. For positive selection of the labelled monocytes from the cell mixture of the PBMC, the entire cell suspension was applied to a column with a ferromagnetic carrier matrix and this was placed in a magnetic field. The cells loaded with microbeads were thereby bound to the carrier matrix, and non-labelled cells passed through the column and were discarded. After removing the matrix from the magnetic field, the antibody-laden cells were eluted by rinsing the now demagnetized column with buffer. The purity of this CD14-positive monocyte population obtained in this way is about 95-98%. These monocytes were incubated in a density of 10⁶ cells/ml of culture medium (RPMI, supplemented with 10% foetal calf serum) with the test substances, dissolved in DMSO, at 37° C. and 5% CO₂ for one hour. 10 μg/ml of LPS from E. coli were then added. After 24 hours, cell-free culture supernatants were taken and tested for the content of IL-10 and IL-12.

The concentration of IL-12 and IL-10 in the cell culture supernatants was determined by means of sandwich ELISAs using two anti-IL-12 and, respectively, IL-10 monoclonal antibodies (Biosource Europe, Fleurus, Belgium). A reference standard curve with human IL-10 and, respectively, IL-12 was included. The detection limit of the ELISAs was 15 pg/ml.

TABLE 1
Influence of the test substances in comparison with thalidomide and the
compound XXVIII (C1 → Comparison Example 1) on the IL-12 and
IL-10 production of LPS-activated monocytes

| | Inhibition of | Increase in |
|---|---|---|
| | IL-12 production | IL-10 production |
| Example no. | IC₅₀ (ng/ml) | EC₂₀₀ (ng/ml) |
| 1 | 1500 | 4000 |
| 2 | 1400 | 1000 |
| 3 | 130 | 400 |
| 4 | 107 | 620 |
| 8b | 7300 | >50000 |
| 8c | 4000 | >50000 |
| 8d | 715 | >50000 |
| 8g | 620 | 1000 |
| 9b | 15000 | 16000 |
| 10a | 303 | 16000 |
| 10c | 1172 | >50000 |
| 11a | 600 | 5000 |
| 11b | 514 | 5600 |
| 12a | 1800 | >50000 |
| 14 | 5706 | 20000 |
| 15b | 4004 | >50000 |
| 15bb | 419 | 5600 |
| 15cc | 6000 | 20000 |
| 15e | 520 | 3000 |
| 15h | 4000 | >50000 |
| 15i | 884 | 15000 |
| 15j | 521 | 5000 |
| 15l | 2000 | 5600 |
| 15u | 1800 | >50000 |
| XXVIII = C1 | >50000 | no increase |
| Thalidomide | 70 | 20000 |

The pyrrolidine(thi)ones substituted by heterocyclic substituents in the 3-position having the basic structure described in formula I suppressed in a potent manner the IL-12 production of LPS-activated monocytes in a concentration-dependent manner. Interestingly, the IL-10 production was increased. The maximum IL-12 inhibition and the IC₅₀ values are significantly above those of compound XXVIII (Comparison Example 1). The most active compounds are those in which the aromatic ring contains a fluoro substitution in position 7 or fluoro substitutions in positions 5 and 7. The action potency is comparable to that of thalidomide.

### Investigations of the Pharmacokinetics

The compound of Example 3 and the piperidine-2,6-dione analogue thereof were dissolved together in a concentration of 5 mg/ml each in physiological saline solution. Volumes of 2 ml/kg were administered intravenously to rats through the tail vein. After defined times, blood samples were taken from the retro-orbital plexus and plasma obtained. Plasma concentrations of both compounds were determined using valid bioanalytical methods. The accompanying drawing FIGURE shows plasma concentration/time curves of Example 3 and the piperidine-2,6-dione analogue thereof following intravenous administration of in each case 10 mg/kg to rats. As can be seen from the FIGURE, the distribution volume of Example 3 was estimated as 0.26 l/kg, and that of the piperidine-2,6-dione analogue as 1.0 l/kg.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A pyrrolidine(thi)one compound substituted by a heterocyclic substituent in the 3-position corresponding to formula (I): 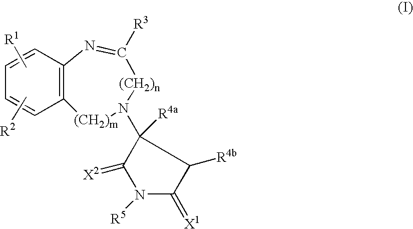
wherein
R¹ and R² are independently selected from the group consisting of H; F; Cl; Br; 1, CN; CF₃; OCF₃; SR; NO₂; branched or unbranched, unsubstituted or mono- or polysubstituted C₁₋₁₀-alkyl, C₂-C₁₀-alkenyl and C₃-C₁₀-alkynyl;
saturated or unsaturated, unsubstituted or mono- or polysubstituted C₃-C₇-cycloalkyl; heterocyclic groups with 2 to 6 ring carbon atoms and one ring member selected from the group consisting of S, O and NR^{5′}; OR^{6′}; OC(O)R^{6′}; OC(S)R^{6′}; C(O)R^{6′}; C(O)OR^{6′}; C(S)R^{6′}; C(S)OR^{6′}; SR^{6′}; S(O)R^{6′}; S(O₂)R^{6′}; NR⁸R⁹; C(O)NR⁸R⁹; and S(O₂)NR⁸R⁹;
wherein
R^{5′} is H or branched or unbranched, unsubstituted or mono- or polysubstituted C₁-C₁₀-alkyl, C₂-C₁₀-alkenyl or C₂-C₁₀-alkynyl;
R^{6′} is selected from the group consisting of H; branched or unbranched, unsubstituted or mono- or polysubstituted C₁-C₁₀-alkyl, C₂-C₁₀-alkenyl or C₃-C₁₀-alkynyl; saturated or unsaturated, unsubstituted or mono- or polysubstituted C₃-C₇-cycloalkyl; heterocyclic groups with 2 to 6 ring carbon atoms and one ring member selected from the group consisting of S, O and NR⁷; saturated or unsaturated, unsubstituted or mono- or polysubstituted alkylaryl; and unsubstituted or mono- or polysubstituted aryl or heteroaryl;
R⁷ is H, or branched or unbranched, unsubstituted or mono- or polysubstituted C₁-C₁₀-alkyl, C₂-C₁₀-alkenyl or C₃-C₁₀-alkynyl;
R⁸ and R⁹ are independently selected from the group consisting of H; branched or unbranched, unsubstituted or mono- or polysubstituted or unsubstituted C₁-C₁₈-alkyl, C₂-C₁₈-alkenyl or C₃-C₁₈-alkynyl;
saturated or unsaturated, unsubstituted or mono- or polysubstituted C₃-C₇-cycloalkyl; heterocyclic groups with 2 to 6 ring carbon atoms and one ring member selected from the group consisting of S, O and NR¹⁰; saturated or unsaturated, unsubstituted or mono- or polysubstituted alkylaryl; and unsubstituted or mono- or polysubstituted aryl or heteroaryl; or
R⁸ and R⁹ together with the nitrogen atom to which they are attached form a 4- to 8-membered, saturated or unsaturated, unsubstituted or mono- or polysubstituted ring in which one C atom optionally may be replaced by S, O or NR¹⁰; and
R¹⁰ is H, or branched or unbranched, unsubstituted or mono- or polysubstituted C₁-C₁₀-alkyl, C₂-C₁₀-alkenyl or C₃-C₁₀-alkynyl;
R³ is selected from the group consisting of H, aryl, heteroaryl, in each case substituted or unsubstituted, C₁-C₁₀-alkyl, C₂-C₁₀-alkenyl or C₃-C₁₀-alkynyl, in each case branched or unbranched, mono- or polysubstituted or unsubstituted; C₃-C₇-cycloalkyl, saturated or unsaturated, mono- or polysubstituted or unsubstituted, or a corresponding heterocyclic radical in which one C atom in the ring is replaced by S, O or NR³,
where R³ is selected from the group consisting of H, C₁-C₁₀-alkyl, C₂-C₁₀-alkenyl or C₂-C₁₀-alkynyl, in each case branched or unbranched, mono- or polysubstituted or unsubstituted,
alkylaryl, saturated or unsaturated, mono- or polysubstituted or unsubstituted; aryl, mono- or polysubstituted or unsubstituted;
or, if a C—N single bond is present, R³ may represent OH, C₁₋₃-alkoxy or an [O(CO)C₁₋₃-alkyl] group, or together with the C atom may represent a carbonyl group;
R^{4a} and R^{4b} each independently denote H, F, alkyl, aryl or heteroaryl, in each case substituted or unsubstituted;
R⁵ represents H, aryl, heteroaryl, in each case substituted or unsubstituted, alkyl, a CH₂—OH group or a CH₂—NR⁶R⁷ group in which R⁶ and R⁷ may be identical or different and denote a straight-chain or branched alkyl group having 1-6 C atoms or together with the N atom denote a pyrrolidine, piperidine, hexamthyleneimine or morpholine ring,
X¹ or X² or both represent O or S, and any remaining X¹ or X² denotes H₂,
n denotes 0 or 1, and
m denotes 1 or 2,
or a salt thereof with a physiologically acceptable acid.

2. A compound according to claim 1, wherein said compound is present in the form of an isolated stereoisomer.

3. A compound according to claim 1, wherein said compound is present in the form of a mixture of stereoisomers in any mixing ratio.

4. A compound according to claim 3, wherein said compound is present in the form of a racemic mixture.

5. A compound according to claim 1, wherein:
R¹ and R² may be identical or different and are independently selected from the group consisting of H, Br, Cl, F, I, CF₃, OH, NO₂, NR⁵R⁶,
where R⁵ and R⁶ are independently selected from the group consisting of H, alkyl and acyl groups;
alkyl, alkoxy, alkylthio, alkylsulfinyl, alkylsulfonyl, aryl, heteroaryl, in each case substituted or unsubstituted, branched or unbranched, or
R¹ and R² together denote a fused-on benzene ring optionally substituted by a further R¹ and R² as defined above;
R³ represents H, a methyl group or, if a C—N single bond is present, together with the C atom may represent a carbonyl group;
R^{4a} and R^{4b} each independently denote H, alkyl, aryl or heteroaryl;
R⁵ represents H, aryl, heteroaryl, in each case substituted or unsubstituted, alkyl, a CH₂—OH group or a CH₂—NR⁶R⁷ group in which R⁶ and R⁷ may be identical or different and denote a straight-chain or branched alkyl group having 1-6 C atoms or together with the N atom to which they are attached denote a pyrrolidine, piperidine, hexamthyleneimine or morpholine ring;
X¹ or X² or both represent O or S, and any remaining X¹ or X² denotes H₂,
n denotes 0 or 1, and
m denotes 1 or 2.

6. A compound according to claim 1, wherein:
said compound contains a C═N double bond;
R¹ and R² may be identical or different and denote H, Br, Cl, F, CF₃, NO₂, NH₂, C₁₋₃-alkyl, or C₁₋₃-alkoxy, or together form a fused-on benzene ring;
R³ represents H or a methyl group;
R^{4a} represents H or a methyl group,
R^{4b} represents H or a phenyl group;
R⁵ represents H or a methyl group;
X¹ and X² each represent O;
n=0, and
m=1.

7. A compound according to claim 1, wherein:
said compound contains a C═N double bond;
R¹ and R² may be identical or different and denote H, Cl or F;
R³, R^{4a}, R^{4b} and R⁵ each denote hydrogen;
X¹ and X² each represent O;
n=0, and
m=1.

8. A compound according to claim 1, selected from the group consisting of:
3-(5-chloro-7-fluoro-4H-quinazolin-3-yl)-pyrrolidine-2,5-dione,
3-(7-chloro-5-fluoro-4H-quinazolin-3-yl)-pyrrolidine-2,5-dione,
3-(7-fluoro-4H-quinazolin-3-yl)-pyrrolidine-2,5-dione and the hydrochloride thereof,
3-(5,7-difluoro-4H-quinazolin-3-yl)-pyrrolidine-2,5-dione and the hydrochloride thereof,
3-(5,7-dichloro-4H-quinazolin-3-yl)-pyrrolidine-2,5-dione,
3-(5-bromo-4H-quinazolin-3-yl)-pyrrolidine-2,5-dione,
3-(7-trifluoromethyl-4H-quinazolin-3-yl)pyrrolidine-2,5-dione,
3-(5,8-dichloro-4H-quinazolin-3-yl)-pyrrolidine-2,5-dione,
3-(5-methyl-4H-quinazolin-3-yl)-pyrrolidine-2,5-dione,
3-(4H-benzo[g]quinazolin-3-yl)-pyrrolidine-2,5-dione,
3-(6,7-difluoro-4H-quinazolin-3-yl)-pyrrolidine-2,5-dione,
3-(6,8-dichloro-4H-quinazolin-3-yl)-pyrrolidine-2,5-dione,
3-(6-nitro-4H-quinazolin-3-yl)-pyrrolidine-2,5-dione,
3-(7-chloro-4H-quinazolin-3-yl)-pyrrolidine-2,5-dione,
3-(7-nitro-4H-quinazolin-3-yl)-pyrrolidine-2,5-dione,
3-(8-bromo-6-methyl-4H-quinazolin-3-yl)-pyrrolidine-2,5-dione,
3-(8-chloro-4H-quinazolin-3-yl)-pyrrolidine-2,5-dione,
3-(8-methoxy-4H-quinazolin-3-yl)-pyrrolidine-2,5-dione,
3-(6-benzyloxy-4H-quinazolin-3-yl)-pyrrolidine-2,5-dione,
3-(5,6-dichloro-4H-quinazolin-3-yl)-pyrrolidine-2,5-dione,
3-(7-methoxy-4H-quinazolin-3-yl)-pyrrolidine-2,5-dione,
3-(5-chloro-4H-quinazolin-3-yl)-pyrrolidine-2,5-dione,
3-(6,7-dimethoxy-4H-quinazolin-3-yl)-pyrrolidine-2,5-dione,
3-(6-chloro-4H-quinazolin-3-yl)-pyrrolidine-2,5-dione,
3-(4H-quinazolin-3-yl)-pyrrolidine-2,5-dione,
3-(5-fluoro-4H-quinazolin-3-yl)-pyrrolidine-2,5-dione,
3-(8-trifluoromethyl-4H-quinazolin-3-yl)-pyrrolidine-2,5-dione,
1-methyl-3-(4H-quinazolin-3-yl)-pyrrolidine-2,5-dione,
7-fluoro-1-methyl-3-(4H-quinazolin-3-yl)-pyrrolidine-2,5-dione,
3-(5-methoxy-4H-quinazolin-3-yl)-pyrrolidine-2,5-dione,
3-(5-ethoxy-4H-quinazolin-3-yl)-pyrrolidine-2,5-dione,
3-(5-pentyloxy-4H-quinazolin-3-yl)-pyrrolidine-2,5-dione,
3-(5-benzyloxy-4H-quinazolin-3-yl)-pyrrolidine-2,5-dione,
3-(5-isopropoxy-4H-quinazolin-3-yl)-pyrrolidine-2,5-dione,
3-(5-(2-methoxyethoxy)-4H-quinazolin-3-yl)-pyrrolidine-2,5-dione,
3-(5-ethanesulfonyl-4H-quinazolin-3-yl)-pyrrolidine-2,5-dione,
3-(5-ethanesulfinyl-4H-quinazolin-3-yl)-pyrrolidine-2,5-dione,
3-(5-ethylthio-4H-quinazolin-3-yl)-pyrrolidine-2,5-dione,
3-(5-nitro-4H-quinazolin-3-yl)-pyrrolidine-2,5-dione,
1-methyl-3-(2-methyl-4H-quinazolin-3-yl)-pyrrolidine-2,5-dione,
3-(2-methyl-4H-quinazolin-3-yl)-pyrrolidine-2,5-dione,
3-(5,6-dichloro-2-methyl-4H-quinazolin-3-yl)-pyrrolidine-2,5-dione,
3-(5,7-dichloro-2-methyl-4H-quinazolin-3-yl)-pyrrolidine-2,5-dione,
3-(5,7-difluoro-2-methyl-4H-quinazolin-3-yl)-pyrrolidine-2,5-dione and the hydrochloride thereof,
3-(5,8-dichloro-2-methyl-4H-quinazolin-3-yl)-pyrrolidine-2,5-dione,
3-(5-benzyloxy-2-methyl-4H-quinazolin-3-yl)-pyrrolidine-2,5-dione,
3-(5-bromo-2-methyl-4H-quinazolin-3-yl)-pyrrolidine-2,5-dione,
3-(5-chloro-2-methyl-4H-quinazolin-3-yl)-pyrrolidine-2,5-dione,
3-(5-chloro-7-fluoro-2-methyl-4H-quinazolin-3-yl)-pyrrolidine-2,5-dione and the hydrochloride thereof,
3-(5-ethoxy-2-methyl-4H-quinazolin-3-yl)-pyrrolidine-2,5-dione,
3-(5-fluoro-2-methyl-4H-quinazolin-3-yl)-pyrrolidine-2,5-dione,
3-(5-isopropoxy-2-methyl-4H-quinazolin-3-yl)-pyrrolidine-2,5-dione,
3-(2,5-dimethyl-4H-quinazolin-3-yl)-pyrrolidine-2,5-dione,
3-(5-methoxy-2-methyl-4H-quinazolin-3-yl)-pyrrolidine-2,5-dione,
3-(2-methyl-5-nitro-4H-quinazolin-3-yl)-pyrrolidine-2,5-dione,
3-(2-methyl-5-pentyloxy-4H-quinazolin-3-yl)-pyrrolidine-2,5-dione,
3-(5-ethylthio-2-methyl-4H-quinazolin-3-yl)-pyrrolidine-2,5-dione,
3-(5-ethanesulfonyl-2-methyl-4H-quinazolin-3-yl)-pyrrolidine-2,5-dione,
3-(2-methyl-4H-benzo[g]quinazolin-3-yl)-pyrrolidine-2,5-dione,
3-(6,7-difluoro-2-methyl-4H-quinazolin-3-yl)-pyrrolidine-2,5-dione,
3-(6,7-dimethoxy-2-methyl-4H-quinazolin-3-yl)-pyrrolidine-2,5-dione,
3-(6,8-dichloro-2-methyl-4H-quinazolin-3-yl)-pyrrolidine-2,5-dione,
3-(6-benzyloxy-2-methyl-4H-quinazolin-3-yl)-pyrrolidine-2,5-dione,
3-(6-chloro-2-methyl-4H-quinazolin-3-yl)-pyrrolidine-2,5-dione,
3-(2-methyl-6-nitro-4H-quinazolin-3-yl)-pyrrolidine-2,5-dione,
3-(2-methyl-7-trifluoromethyl-4H-quinazolin-3-yl)-pyrrolidine-2,5-dione,
3-(7-chloro-2-methyl-4H-quinazolin-3-yl)-pyrrolidine-2,5-dione,
3-(7-fluoro-2-methyl-4H-quinazolin-3-yl)-pyrrolidine-2,5-dione,
3-(7-methoxy-2-methyl-4H-quinazolin-3-yl)-pyrrolidine-2,5-dione,
3-(2-methyl-7-nitro-4H-quinazolin-3-yl)-pyrrolidine-2,5-dione,
3-(8-bromo-2,6-dimethyl-4H-quinazolin-3-yl)-pyrrolidine-2,5-dione,
3-(2-methyl-8-trifluoromethyl-4H-quinazolin-3-yl)-pyrrolidine-2,5-dione,
3-(8-chloro-2-methyl-4H-quinazolin-3-yl)-pyrrolidine-2,5-dione,
3-(8-methoxy-2-methyl-4H-quinazolin-3-yl)-pyrrolidine-2,5-dione,
3-(5-(2-methoxyethoxy)-2-methyl-4H-quinazolin-3-yl)pyrrolidine-2,5-dione,
3-(7-amino-4H-quinazolin-3-yl)-pyrrolidine-2,5-dione hydrochloride,
3-(7-amino-2-methyl-4H-quinazolin-3-yl)-pyrrolidine-2,5-dione hydrochloride,
3-(6-amino-4H-quinazolin-3-yl)-pyrrolidine-2,5-dione hydrochloride,
3-(6-amino-2-methyl-4H-quinazolin-3-yl)-pyrrolidine-2,5-dione hydrochloride,
3-(5-amino-4H-quinazolin-3-yl)-pyrrolidine-2,5-dione hydrochloride,
3-(5-amino-2-methyl-4H-quinazolin-3-yl)-pyrrolidine-2,5-dione hydrochloride,
3-(5-hydroxy-4H-quinazolin-3-yl)-pyrrolidine-2,5-dione hydrochloride,
3-(6-hydroxy-2-methyl-4H-quinazolin-3-yl)-pyrrolidine-2,5-dione hydrochloride,
3-(5-hydroxy-2-methyl-4H-quinazolin-3-yl)-pyrrolidine-2,5-dione hydrochloride,
3-(5-hydroxy-2-methyl-4H-quinazolin-3-yl)-pyrrolidine-2,5-dione hydrochloride,
3-(7-fluoro-4H-quinazolin-3-yl)-5-thioxopyrrolidin-2-one hydrobromide,
3-(4H-quinazolin-3-yl)-5-thioxopyrrolidin-2-one hydrobromide,
3-methyl-3-(4H-quinazolin-3-yl)-pyrrolidine-2,5-dione,
3-(5-bromo-4H-quinazolin-3-yl)-3-methyl-pyrrolidine-2,5-dione,
3-(2-oxo-1,4-dihydro-2H-quinazolin-3-yl)-pyrrolidine-2,5-dione,
3-(7-fluoro-2-oxo-1,4-dihydro-2H-quinazolin-3-yl)-pyrrolidine-2,5-dione,
3-(2-oxo-1,4-dihydro-2H-quinazolin-3-yl)-4-phenylpyrrolidine-2,5-dione,
3-(2-thio-1,4-dihydro-2H-quinazolin-3-yl)-pyrrolidine-2,5-dione,
3-(2-(methylthio)-4H-quinazolin-3-yl)pyrrolidine-2,5-dione hydroiodide,
3-(2-(dimethylamino)-4-quinazolin-3-yl)pyrrolidine-2,5-dione hydroiodide, and
3-(4H-quinazolin-3-yl)-pyrrolidin-2-one.

9. A process for preparing a pyrrolidine(thi)one compound substituted by a heterocyclic substituent in the 3-position according to claim 1, said process comprising:
a) for the preparation of a compound in which X¹ and X² are oxygen, reacting a 2-aminobenzylamine compound of formula (II): 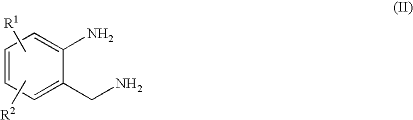
in which R¹ and R² are as defined in claim 1,
with a pyrrole-2,5-dione compound of formula (III): 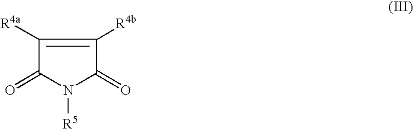
in which R^{4a} and R^{4b} are as defined in claim 1,
to give an amine corresponding to formula (IVa): 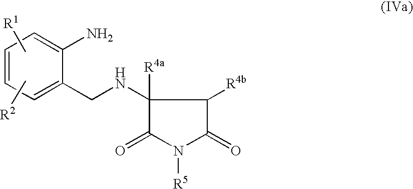
wherein Instead of the pyrrole-2,5-diones, 3-bromo-pyrrolidine-2,5-diones of the general formula (IIIa) 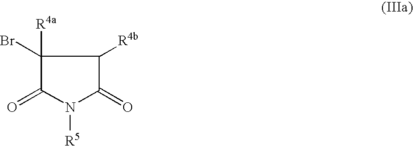
are optionally employed, or
b) for the preparation of a compound in which at least one of X¹ and X² denotes sulfur
a compound of formula (IVb) in which at least one 0 from formula (IVa) is replaced by S 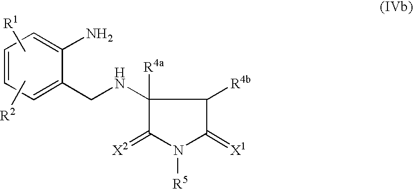
are prepared from (IVa),
optionally after protecting the two nitrogen atoms which are not bonded to R⁵, by exchange of at least one O for X¹ or X² as S with a thionation reagents, and subsequent deprotection,
and subsequently reacting compound (IVa) or (IVb)
with a compound of formula (V)
R³—C(OR^{y})₃ (V)
wherein R³ denotes hydrogen or a methyl group, and
R^{y} represents a straight-chain or branched C₁-C₄-alkyl group,
or with an amidine salt of formula (VI): 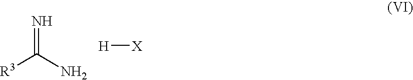
wherein R³ is as defined above and X represents the anion of an acid,
or with a carboxylic acid ester of formula (Vb): 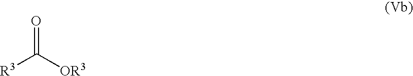
and then preparing a compound of formula (I) with the corresponding meaning of R³ from a compound of formula (IVa) or from compound of formula (IVb).

10. A process according to claim 9, wherein a compound of formula (I) in which R³ together with the C atom to which R³ is attached represents a germinally substituted C atom is prepared by reacting a compound of formula (IVa) or (IVb) with a ketone of formula (Vc): 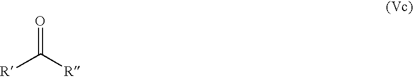
wherein R′ and R″ independently represent an alkyl, aryl or heteroaryl group.

11. A process according to claim 9, wherein a compound of formula (I) in which R³ together with the C atom to which it is attached represent a carbonyl group is prepared:
by reacting a compound of formula (IVa) or (IVb) with C1 units of formula (VII) 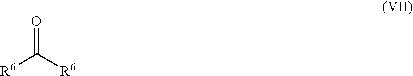
wherein R⁶ represents Cl, an imidazol-1-yl group, a C₁-C₄-alkoxy group, a phenyloxy group, a phenyloxy group substituted by a nitro group, chlorine or fluorine, or a thiomethyl group, or
by reacting a compound of formula (IVa) or (IVb) with a C₁-C₄-alkyl ester, a phenyl ester, or a 4-nitro-, 4-chloro- or 4-fluoro-substituted phenyl ester of chloroformic acid.

12. A process according to claim 9, wherein a compound of formula (I) in which R³ together with the C atom to which it is attached represent a carbonyl group is prepared by reacting a compound of formula (IVa) or (IVb) with a C1 unit of formula VIII:
C(OR⁷)₄ (VIII)
in which R⁷ represents a methyl or ethyl group.

13. A process according to claim 9, wherein an aminobenzylamine of formula (II) is reacted with a pyrrole-2,5-dione of formula (III) in an inert solvent at room temperature.

14. A process according to claim 9, wherein a compound of formula (Iva) or (IVb) is reacted with a compound of formula (V) either without a solvent or in an organic carboxylic acid in a temperature range of from 10 to 150° C.

15. A process for preparing a pyrrolidine(thi)one compound substituted by a heterocyclic substituent in the 3-position according to claim 1, said process comprising initially alkylating an amino compound of formula (IX): 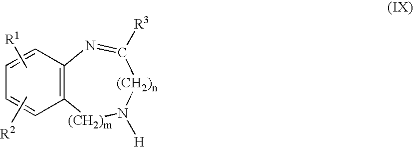
with a pyrrole-2,5-dione of formula (III) or a 3-bromopyrrolidine-2,5-dione derivative of formula (IIIa) 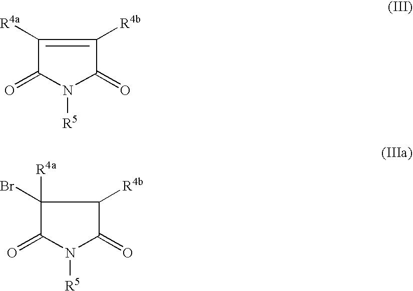
to give a compound of formula (Ia) 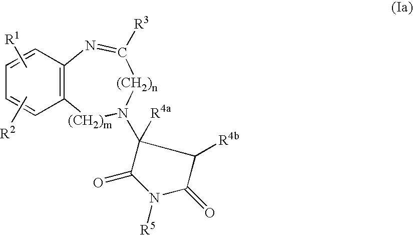
wherein in the compounds (Ia), (II) and (III) the radicals R¹ to R^{4b} are as defined in claim 1, and
if R⁵ is not hydrogen, then R⁵ is subsequently introduced by reaction with formaldehyde, optionally together with an amine of formula HNR⁶R⁷, wherein
R⁶ and R⁷ are as defined in claim 1, and
optionally sulfurizing X¹ or X² or both.

16. A process for the preparing a pyrrolidine(thi)one compound substituted by a heterocyclic substituent in the 3-position according to claim 1, said process comprising:
initially alkylating an amino compound of formula (II) with a 3-bromopyrrolidin-2-one compound of formula (X): 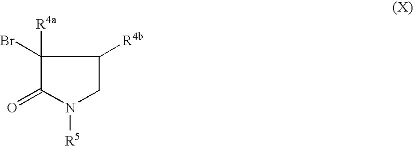
to give a compound of formula (Ib): 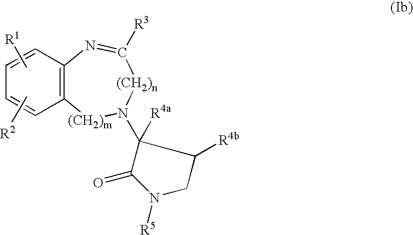
and subsequently oxidizing the compound of formula (Ib) to give a compound of formula (Ia), and
optionally introducing other substituents R⁴ or R⁵ or both.

17. A process for preparing a pyrrolidine(thi)one compound substituted by a heterocyclic substituent in the 3-position according to claim 1, in which m=1; n=0, and R³ represents H or OH,
said process comprising initially oxidizing a formamide compound of formula (XXIII) 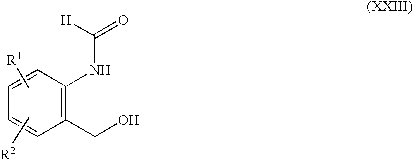
wherein R¹ and R² are as defined in claim 1, to give a benzaldehyde of formula (XXIV): 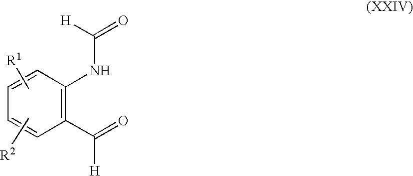
and converting the benzaldehyde of formula (XXIV) by reductive amination with asparagine or corresponding derivatives having a radical R⁴ which is not H using complex borohydrides into a compound of formula (XXV): 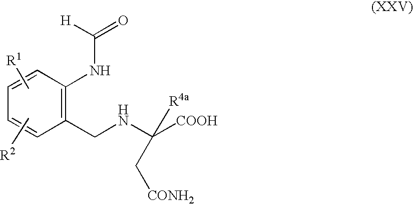
and cyclizing the compound of formula (XXV), optionally after protecting the amine function, and then subsequently eliminating the protecting group, to give a succinimide of formula (XXVI): 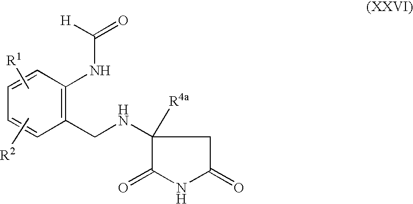
from which a compound according to claim 1, in which R¹, R² and R^{4a} are as defined in claim 1, and m represents 1, n represents 0, a C═N double bond is present, and R³ and R⁵ represent a hydrogen atom, which can optionally be exchanged for other substituents R⁵ as defined in claim 1, is obtained by acid catalysis in a protic solvent, and
after the reductive amination of the compound of formula (XXIV), optionally treating the reaction mixture with an acid to give a compound of formula (XXVII) in which R¹, R² and R⁴ are as defined above 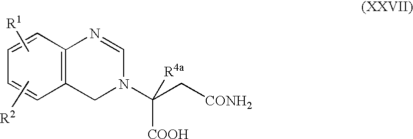
and cyclizing the compound of formula (XXVII) to convert it to a compound of formula (I) in which m=1 and n=0, a C═N double bond is present, R¹, R² and R⁴ are as defined in claim 1, and R³ and R⁵ represent hydrogen,
and optionally subsequently introducing other substituents R⁴ or R⁵ or both as defined in claim 1.

18. A pharmaceutical composition comprising a compound according to claim 1, and at least one pharmaceutically acceptable carrier or auxiliary substance.

19. A method for treating or inhibiting an inflammatory or autoimmune or haematological-oncological disease state in a subject in need thereof, said method comprising administering to said subject a pharmacologically effective amount of a compound according to claim 1.

20. A method of modulating autoimmune activity in a subject in need thereof, said method comprising administering to said subject an effective autoimmune activity modulating amount of a compound according to claim 1.
